SEQUENCE LISTING (50 sequences)

SEQ ID NO: 1 - RNA, 19 nt, Artificial Sequence
  Synthetic primer
uuugauuuugucuaaaacc

SEQ ID NO: 2 - RNA, 19 nt, Artificial Sequence
  Synthetic primer
uuugauuuugucuaaaacc

SEQ ID NO: 3 - RNA, 19 nt, Artificial Sequence
  Synthetic primer
uuugauuuugacuaaaacc

SEQ ID NO: 4 - RNA, 19 nt, Artificial Sequence
  Synthetic primer
gauuuugucaaaaacccug

SEQ ID NO: 5 - RNA, 19 nt, Artificial Sequence
  Synthetic primer
ucaaggaaguuggcauuuc

SEQ ID NO: 6 - RNA, 19 nt, Artificial Sequence
  Synthetic primer
caucaaggaacauggcauu

SEQ ID NO: 7 - DNA, 24 bp, Artificial Sequence
  Synthetic primer
ttgatatgtggatttcgagtcgtc

SEQ ID NO: 8 - DNA, 28 bp, Artificial Sequence
  Synthetic primer
cctcccatatgtccagattctcttgatg

SEQ ID NO: 9 - DNA, 25 bp, Artificial Sequence
  Synthetic primer
cagatggtttttcaaaatagagtcc

SEQ ID NO: 10 - DNA, 29 bp, Artificial Sequence
  Synthetic primer
aaaacgatcctgagacttccacactgatg

SEQ ID NO: 11 - RNA, 19 nt, Artificial Sequence
  Synthetic primer
cuuaccucaguuacaauuu

SEQ ID NO: 12 - RNA, 19 nt, Artificial Sequence
  Synthetic primer
cucuuaccucaguuacaau

SEQ ID NO: 13 - RNA, 19 nt, Artificial Sequence
  Synthetic primer
accucuuaccucaguuaca

SEQ ID NO: 14 - RNA, 19 nt, Artificial Sequence
  Synthetic primer
aaaccucuuaccucaguua

SEQ ID NO: 15 - RNA, 19 nt, Artificial Sequence
  Synthetic primer
ugaaaccucuuaccucagu

SEQ ID NO: 16 - RNA, 19 nt, Artificial Sequence
  Synthetic primer
cuuaccucagauacaauuu

SEQ ID NO: 17 - RNA, 19 nt, Artificial Sequence
  Synthetic primer
cucuuaccucuguuacaau

SEQ ID NO: 18 - RNA, 19 nt, Artificial Sequence
  Synthetic primer
accucuuaccacaguuaca

SEQ ID NO: 19 - RNA, 19 nt, Artificial Sequence
  Synthetic primer
aaaccucuuagcucaguua

SEQ ID NO: 20 - RNA, 19 nt, Artificial Sequence
  Synthetic primer
ugaaaccucuaaccucagu

SEQ ID NO: 21 - RNA, 19 nt, Artificial Sequence
  Synthetic primer
ugaaagcucuuaccucagu

SEQ ID NO: 22 - RNA, 19 nt, Artificial Sequence
  Synthetic primer
ugaaaccugaaaccucagu

SEQ ID NO: 23 - RNA, 19 nt, Artificial Sequence
  Synthetic primer
gcuauaccagcgucgucau

SEQ ID NO: 24 - RNA, 19 nt, Artificial Sequence
  Synthetic primer
uagcuuuauauggauguua

SEQ ID NO: 25 - RNA, 19 nt, Artificial Sequence
  Synthetic primer
uaaaacccuguaaggaaaa

SEQ ID NO: 26 - RNA, 19 nt, Artificial Sequence
  Synthetic primer
ucuaaaacccuguaaggaa

SEQ ID NO: 27 - RNA, 19 nt, Artificial Sequence
  Synthetic primer
cuuucauaaugcuggcaga

SEQ ID NO: 28 - RNA, 19 nt, Artificial Sequence
  Synthetic primer
ucacuuucauaaugcuggc

SEQ ID NO: 29 - RNA, 19 nt, Artificial Sequence
  Synthetic primer
cuuucuaacaucugaacuu

SEQ ID NO: 30 - RNA, 19 nt, Artificial Sequence
  Synthetic primer
caacuuucuaacaucugaa

SEQ ID NO: 31 - RNA, 19 nt, Artificial Sequence
  Synthetic primer
uagcuuuauuuggauguua

SEQ ID NO: 32 - RNA, 19 nt, Artificial Sequence
  Synthetic primer
guuucacaacacauuuuac

SEQ ID NO: 33 - RNA, 19 nt, Artificial Sequence
  Synthetic primer
uuuugucuaauacccugua

SEQ ID NO: 34 - RNA, 19 nt, Artificial Sequence
  Synthetic primer
aggaaugugaccaccuucc

SEQ ID NO: 35 - RNA, 19 nt, Artificial Sequence
  Synthetic primer
cuuucauaaagcuggcaga

SEQ ID NO: 36 - RNA, 19 nt, Artificial Sequence
  Synthetic primer
ucacuuucaaaaugcuggc

SEQ ID NO: 37 - RNA, 19 nt, Artificial Sequence
  Synthetic primer
cuuucuaacuucugaacuu

SEQ ID NO: 38 - RNA, 19 nt, Artificial Sequence
  Synthetic primer
ggaagauggcuuuucuagu

SEQ ID NO: 39 - RNA, 19 nt, Artificial Sequence
  Synthetic primer
aaggaagaugccauuucua

SEQ ID NO: 40 - RNA, 19 nt, Artificial Sequence
  Synthetic primer
ucaaggaagaaggcauuuc

SEQ ID NO: 41 - RNA, 19 nt, Artificial Sequence
  Synthetic primer
aacaucaagguagauggca

SEQ ID NO: 42 - RNA, 19 nt, Artificial Sequence
  Synthetic primer
gccgccgacgcauucuugg

SEQ ID NO: 43 - RNA, 19 nt, Artificial Sequence
  Synthetic primer
uugccgccgucccauucuu

SEQ ID NO: 44 - RNA, 19 nt, Artificial Sequence
  Synthetic primer
ucuugccgcggacccauuc

SEQ ID NO: 45 - RNA, 19 nt, Artificial Sequence
  Synthetic primer
cgccgacccuuucuuggug

SEQ ID NO: 46 - DNA, 52 bp, Artificial Sequence
  Synthetic primer
gaggagaacggcggctgggtaagaaccacgccccttgtgtgtccgccccttg

SEQ ID NO: 47 - DNA, 34 bp, Artificial Sequence
  Synthetic primer
aggcaatggtgccgaggacagtaggtatgactca

SEQ ID NO: 48 - DNA, 20 bp, Artificial Sequence
  Synthetic primer
acctactgtcctcggcacca

SEQ ID NO: 49 - DNA, 20 bp, Artificial Sequence
  Synthetic primer
ctactgtcctcggcaccatt

SEQ ID NO: 50 - DNA, 20 bp, Artificial Sequence
  Synthetic primer
atacctactgtcctcggcac

(19) United States
(12) Patent Application Publication
(10) Pub. No.: US 2014/0128449 A1
(43) Pub. Date: May 8, 2014

(54) OLIGONUCLEOTIDE MODULATION OF SPLICING

(71) Applicants: Jing Liu, Dallas, TX (US); Jiaxin Hu, Dallas, TX (US); David R. Corey, Dallas, TX (US)

(72) Inventors: Jing Liu, Dallas, TX (US); Jiaxin Hu, Dallas, TX (US); David R. Corey, Dallas, TX (US)

(73) Assignee: The Board of Regents of the University of Texas System, Austin, TX (US)

(21) Appl. No.: 14/009,745

(22) PCT Filed: Mar. 23, 2012
(86) PCT No.: PCT/US12/30271

§ 371 Date: Jan. 10, 2014

Related U.S. Application Data
(60) Provisional application No. 61/472,957, filed on Apr. 7, 2011.

(51) Int. Cl. C12N 15/113

(52) U.S. Cl. CPC C12N 15/113; C12N 15/1138; C12N 15/1135
USPC 514/44 A; 435/375; 536/24.5

(57) ABSTRACT

The present invention relates to the selective modulation of pre-mRNA splicing, in particular, for that involving alternative splicing in disease-related proteins such as those involved in Duschenne's Muscular Dystropy and Spinal Muscular Atrophy.

This application claims benefit of priority to U.S. Provisional Application Ser. No. 61/472,957, filed Apr. 7, 2011, the entire contents of which are hereby incorporated by reference.

### STATEMENT REGARDING FEDERALLY SPONSORED RESEARCH OR DEVELOPMENT

This invention was made in part with government support under Grant Nos. RO1-GM073042 and GM077253 awarded by the National Institutes of Health. The United States Government has certain rights in the invention.

### BACKGROUND OF THE INVENTION

A. Field of the Invention

The invention relates to the fields of biology and medicine. More particularly, the invention provides compositions and methods for the modulation of splicing, such as for that are aberrantly/alternatively spliced in disease states.

B. Related Art

Newly synthesized transcripts (pre-mRNAs) contain intervening sequences (introns). These introns must be excised from the pre-mRNA by the spliceosome, a ribonucleoprotein complex. The remaining portions of the pre-mRNA (exons) are then spliced to form the mature mRNA that codes for proteins. Splicing occurs in the nucleus and spliced transcripts are exported into the cytoplasm. Splicing usually does not produce a single mRNA species for each gene. Instead, pre-mRNAs are spliced in alternate ways, leading to production of different proteins. This phenomenon is known as alternative splicing and is observed in over 90% of all human genes (Keren et al., 2010).

Approximately 60% of disease-causing point mutations are related to defective splicing (Jensen et al., 2009) and chemical agents that redirect splicing may promote production of protein isoforms to compensate for genetic defects (Bauman et al., 2009; Aartsma-Rus and van Ommen, 2007; Wood et al., 2010). For example, Duchenne muscular dystrophy is an incurable disease caused by mutations in the DMD gene encoding dystrophin protein (Lu et al., 2010). Agents that promote alternative splicing might cause the mutated region to be deleted, leading to production of a truncated version of dystrophin that is naturally found in patients suffering from a more mild disease, Becker's muscular dystrophy. Induction of truncated dystrophin might convert a fatal genetic disease into a condition where patients experience a normal lifespan and a good quality of life.

### SUMMARY OF THE INVENTION

Thus, in accordance with the present invention, there is provided a method for modulating splicing of a pre-mRNA comprising contacting a cell that produces a pre-mRNA with alternative splicing with a double-stranded RNA of 15-30 bases that targets a splice junction or exonic or intronic sequences adjacent thereto, wherein (a) said alternative splicing produces an “exon-included” product and said double-stranded RNA contains one or more centrally located mismatches; or (b) said alternative splicing produces and “exon-excluded” product and said double-stranded RNA is fully complementary to a target site. The pre-mRNA may encodea protein that is aberrantly spliced in a disease state, such as the Duchenne muscular dystrophy protein, survival motor neuron 2, the APOB protein, the Bcl-x protein or the insulin receptor protein. In particular, the double-stranded RNA operates through an AGO2-dependent mechanism.

The double-stranded RNA may be about 19 to about 21 bases in length. The double-stranded RNA comprises one or more chemically-modified bases, such as a 2′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, or a non-natural base comprising nucleotide. The guide strand or the passenger strand or both may contain the chemically-modified base(s).

The double-stranded RNA may comprise at least 15 bases and the first base mismatch is flanked by at least 7 bases on both the 5′ and 3′ ends of the double-stranded RNA. The first base mismatch may be in the guide strand. The double-stranded RNA may further comprise a second base mismatch, such as in the passenger strand. The first and second base mismatches may be in the guide strand, and further, the double-stranded RNA may comprise at least 16 bases and the first and second base mismatches are adjacent and flanked by at least 7 bases on both the 5′ and 3′ ends of the double-stranded RNA. Alternatively, the first and second mismatches may be in the passenger strand, and further the double-stranded RNA may comprise at least 16 bases and the first and second base mismatches are adjacent and flanked by at least 7 bases on both the 5′ and 3′ ends of the double-stranded RNA. The double-stranded RNA may be at least 19 bases in length and further comprise a third base mismatch in the guide strand, where the first and second mismatches may be in the guide strand or the passenger strand. The double-stranded RNA may be at least 19 bases in length and further comprise a third base mismatch in the passenger strand, where the first and second mismatches may be in the guide strand or the passenger strand. The dsRNA may have the sequence UUUGAUUUUGUCUAAAACC (SEQ ID NO:1), GAUUUUGUCUAAAACCCUG (SEQ ID NO:2), UUUGAUUUUGACUAAAACC (SEQ ID NO:3), GAUUUUGUCAAAAACCCUG (SEQ ID NO:4), UCAAGGAAGUUGGCAUUUC (SEQ ID NO:5) or CAUCAAGGAACAUGGCAUU (SEQ ID NO:6).

The double-stranded RNA may target a splice junction sequence, an exonic sequence flanking a splice junction sequence, an intronic sequence flanking a splice junction sequence, or any combination thereof. The splicing of the pre-mRNA may be shifted such that an alternative exon is included. The splicing of the pre-mRNA may be shifted such than an alternative exon is excluded. The double-stranded RNA may comprise a non-natural internucleotide linkage, such as a phosphorothioate linkage. The non-natural internucleotide linkage may be in a guide strand, a passenger strand or both.

In another embodiment, there is provided a method for modulating splicing, in a subject, of an pre-mRNA encoding a disease protein and having alternative splicing comprising administering to the subject a double-stranded RNA of 15-30 bases that targets a splice junction or exonic or intronic sequences adjacent thereto, wherein (a) said alternative splicing produces an “exon-included” product and said double-stranded RNA contains one or more centrally located mismatches; or (b) said alternative splicing produces and “exon-excluded” product and said double-stranded RNA is fully complementary to a target site. The subject may be a human or non-human mammal. The pre-mRNA may encode a protein that is aberrantly spliced in a disease state, such as the Duchenne muscular dystrophy protein, survival motor neuron 2, the APOB protein, the Bcl-x protein or the insulin receptor protein. In particular, the double-stranded RNA operates through an AGO2-dependent mechanism.

The double-stranded RNA may be about 19 to about 21 bases in length. The double-stranded RNA comprises one or more chemically-modified bases, such as a 2′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, or a non-natural base comprising nucleotide. The guide strand or the passenger strand or both may contain the chemically-modified base(s).

The double-stranded RNA may comprise at least 15 bases and the first base mismatch is flanked by at least 7 bases on both the 5′ and 3′ ends of the double-stranded RNA. The first base mismatch may be in the guide strand. The double-stranded RNA may further comprise a second base mismatch, such as in the passenger strand. The first and second base mismatches may be in the guide strand, and further, the double-stranded RNA may comprise at least 16 bases and the first and second base mismatches are adjacent and flanked by at least 7 bases on both the 5′ and 3′ ends of the double-stranded RNA. Alternatively, the first and second mismatches may be in the passenger strand, and further the double-stranded RNA may comprise at least 16 bases and the first and second base mismatches are adjacent and flanked by at least 7 bases on both the 5′ and 3′ ends of the double-stranded RNA. The double-stranded RNA may be at least 19 bases in length and further comprise a third base mismatch in the guide strand, where the first and second mismatches may be in the guide strand or the passenger strand. The double-stranded RNA may be at least 19 bases in length and further comprise a third base mismatch in the passenger strand, where the first and second mismatches may be in the guide strand or the passenger strand. The dsRNA may have the sequence UUUGAUUUUGUCUAAAACC (SEQ ID NO:1), GAUUUUGUCUAAAACCCUG (SEQ ID NO:2), UUUGAUUUUGACUAAAACC (SEQ ID NO:3), GAUUUUGUCAAAAACCCUG (SEQ ID NO:4), UCAAGGAAGUUGGCAUUUC (SEQ ID NO:5) or CAUCAAGGAACAUGGCAUU (SEQ ID NO:6).

The double-stranded RNA may target a splice junction sequence, an exonic sequence flanking a splice junction sequence, an intronic sequence flanking a splice junction sequence, or any combination thereof. The splicing of the pre-mRNA may be shifted such that an alternative exon is included. The splicing of the pre-mRNA may be shifted such than an alternative exon is excluded. The double-stranded RNA may comprise a non-natural internucleotide linkage, such as a phosphorothioate linkage. The non-natural internucleotide linkage may be in a guide strand, a passenger strand or both.

The double-stranded RNA may be administered more than once. The double-stranded RNA may be administered by oral or parenteral routes, including intracranial (including intraparenchymal and intraventricular), intrathecal, epidural, intravenous, intraarterial, intramuscular, intraperitoneal, subcutaneous, transdermal, airway (aerosol), nasal, rectal, vaginal and topical (including buccal and sublingual) administration. The double-stranded RNA may be administered in a saline formulation and/or in a lipid formulation. The method may further comprise administering a second therapy to the subject.

In still another embodiment, there is provided a composition of matter comprising a double-stranded RNA of 15-30 bases that that targets a splice junction or exonic or intronic sequences adjacent thereto in a pre-mRNA, wherein (a) said alternative splicing produces an “exon-included” product and said double-stranded RNA contains one or more centrally located mismatches; or (b) said alternative splicing produces and “exon-excluded” product and said double-stranded RNA is fully complementary to a target site. The pre-mRNA may encode a protein that is aberrantly spliced in a disease state, such as the Duchenne muscular dystrophy protein, survival motor neuron 2, the APOB protein, the Bcl-x protein or the insulin receptor protein. In particular, the double-stranded RNA operates through an AGO2-dependent mechanism.

The double-stranded RNA may be about 19 to about 21 bases in length. The double-stranded RNA comprises one or more chemically-modified bases, such as a 2′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, or a non-natural base comprising nucleotide. The guide strand or the passenger strand or both may contain the chemically-modified base(s).

The double-stranded RNA may comprise at least 15 bases and the first base mismatch is flanked by at least 7 bases on both the 5′ and 3′ ends of the double-stranded RNA. The first base mismatch may be in the guide strand. The double-stranded RNA may further comprise a second base mismatch, such as in the passenger strand. The first and second base mismatches may be in the guide strand, and further, the double-stranded RNA may comprise at least 16 bases and the first and second base mismatches are adjacent and flanked by at least 7 bases on both the 5′ and 3′ ends of the double-stranded RNA. Alternatively, the first and second mismatches may be in the passenger strand, and further the double-stranded RNA may comprise at least 16 bases and the first and second base mismatches are adjacent and flanked by at least 7 bases on both the 5′ and 3′ ends of the double-stranded RNA. The double-stranded RNA may be at least 19 bases in length and further comprise a third base mismatch in the guide strand, where the first and second mismatches may be in the guide strand or the passenger strand. The double-stranded RNA may be at least 19 bases in length and further comprise a third base mismatch in the passenger strand, where the first and second mismatches may be in the guide strand or the passenger strand. The dsRNA may have the sequence UUUGAUUUUGUCUAAAACC (SEQ ID NO:1), GAUUUUGUCUAAAACCCUG (SEQ ID NO:2), UUUGAUUUUGACUAAAACC (SEQ ID NO:3), GAUUUUGUCAAAAACCCUG (SEQ ID NO:4), UCAAGGAAGUUGGCAUUUC (SEQ ID NO:5) or CAUCAAGGAACAUGGCAUU (SEQ ID NO:6).

The double-stranded RNA may target a splice junction sequence, an exonic sequence flanking a splice junction sequence, an intronic sequence flanking a splice junction sequence, or any combination thereof. The double-stranded RNA may shift splicing of the pre-mRNA such that an alternative exon is included. The double-stranded RNA may shift splicing of the pre-mRNA such that an alternative exon is excluded. The double-stranded RNA may comprise a non-natural internucleotide linkage, such as a phosphorothioate linkage. The non-natural internucleotide linkage may be in a guide strand, a passenger strand or both. The double-stranded RNA may be administered in a saline formulation and/or in a lipid formulation.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to these drawings and the detailed description presented below.

FIGS. 1A-G. Duplex RNAs alter splicing of Luciferase pre-mRNA. (FIG. 1A) Schematic of engineered luciferase gene showing target region for duplex RNAs. (FIG. 1B) PCR amplification followed by gel electrophoresis to separate aberrant and correct splice products upon addition of duplex RNAs. (FIG. 1C) Increase in luciferase activity upon addition of duplex RNAs. (FIGS. 1D-E) Effect on splicing and luciferase activity from addition of increasing concentrations of duplex RNA 709. (FIG. 1F) PCR amplification followed by gel electrophoresis to separate aberrant and correct splice products after addition of single mismatch-containing RNA duplexes. (FIG. 1G) Effect on splicing of multiple or seed sequence mismatches within RNA duplexes based on RNA 709. Duplex RNAs were transfected into HeLa-derived pLUC/705 cells at 50 nM unless otherwide noted.

FIGS. 2A-J. Mechanism of duplex RNAs that alter splicing of luciferase pre-mRNA. (FIG. 2A) Effect of duplex RNAs on levels of luciferase mRNA measured by quantitative PCR. (FIG. 2B) Localization of AGO1 and AGO2 in purified nuclei from HeLa pLuc/705 cells. (FIG. 2C) Effect of siRNA-mediated reduction of AGO1 or AGO2 levels on the ability of RNAs 709 and 709M11 to enhance luciferase expression. (FIG. 2D) RNA immunoprecipitation (RIP) to show recruitment of AGO1 or AGO2 to Luc/705 pre-mRNA. RNA levels were measured by qPCR. (FIGS. 2E-F) Effect of mismatches on either the guide or passenger strands. (FIG. 2G) Effect of siRNA-mediated reduction of HP1a levels on the ability of RNAs 709 and 709M11 to enhance luciferase expression. (FIG. 2H) Effect of adding TSA and/or 5-AZA-C on the ability of duplex RNA 709 to mediate increased luciferase activity. (FIGS. 2I-J) Chromatin immunoprecipitation (ChIP) showing effect of RNA 709 on H3K9me2 or H3K27me3 modification, evaluated by primer sets designed to amplify three different regions of the gene. Splice correction was calculated as a percentage of the total amount of spliced mRNA, i.e., correct mRNA*100/(correct mRNA+aberrant mRNA). Unless otherwise noted, duplex RNAs were transfected into HeLa-derived pLUC/705 cells at 50 nM.

FIGS. 3A-I. Effect of duplex RNAs on splicing of SMN2. (FIG. 3A) Schematic of splicing of exons 6, 7, and 8. The dark bar within exon 7 represents the position of the CT transition in SMN2 relative to SMN1. (FIGS. 3B-C) Semiquantitive RT-PCR showing effect of fully complementary duplex RNAs (100 nM) on splicing of SMN2. (FIGS. 3D-E) Semiquantitive RT-PCR showing effect of mismatch-containing complementary duplex RNAs (100 nM) on splicing of SMN2. (FIGS. 3F-G) Effect of varying concentrations of mismatch-containing duplexes E01-19 or I03-E16 on splicing of SMN2 pre-mRNA. (FIGS. 3H-I) Comparison of mismatch and full complementary duplexes. Experiments were performed in SMA type I fibroblast GM03813 cells were transfected with RNA duplexes for 24 hr. ISS: intronic splicing silencer; ESS: exonic splicing silencer.

FIG. 4. Effect of duplex RNAs on splicing of dystrophin. RNA sequences are listed in Table 3. All duplexes contain a single mismatch at position 9, except 68M10 which contains a mismatch at position 10.

FIGS. 5A-G. Quantitation of data in FIGS. 1A-G and data used for determination of IC₅₀ values. (FIG. 5A) Quantitation of data shown in FIG. 1A. (FIGS. 5B-C) Effect on splicing of increase concentrations of 2′-O-methyl single stranded oligonucleotide 705Me, and mismatch-containing duplex RNA 709M11. Splicing correction was calculated as a percentage of the total amount of spliced mRNA, i.e., correct mRNA*100/(correct mRNA+aberrant mRNA). (FIGS. 5E-F) Quantitation of data in FIGS. 1F-G. Splicing correction was calculated as a percentage of the total amount of spliced mRNA, i.e., correct mRNA*100/(correct mRNA+aberrant mRNA). (FIGS. 5G-H) Relative luciferase activity measured for treatments related to FIGS. 1F-G.

FIGS. 6A-C. Efficiency of gene knockdown by siRNAs targeting mRNA. Knockdown of mRNAs encoding (FIG. 6A) AGO1, AGO2, or (FIG. 6B) HP1a are shown. (FIG. 6C) Quantitation of data in FIG. 2E showing effect of placing mutations within either the guide and/or passenger strand of duplex 709.

FIGS. 7A-E. (FIG. 7A) Quantitation of FIG. 3A showing percentage of exon 7 inclusion. (FIGS. 7B-C) Effect of on splicing of SMN2 of addition duplex RNAs E01-E19 or I03-E16 at increasing concentrations (quantitation of data is shown in FIGS. 2D-E). (FIGS. 7D-E) Quantitation showing decrease of exon 7 inclusion.

FIGS. 8A-E. (FIG. 8A) Effect of AGO1 or AGO2 knockdown on RNA-mediated alteration of splicing. (FIG. 8B) Use of siRNAs to knockdown AGO1 or AGO2 in SMA type I GM03813 fibroblast cells. (FIG. 8C) Effect of HP1a knockdown on RNA-mediated alteration of splicing. (FIG. 8D) Use of siRNAs to knockdown HP1a in SMA type I GM03813 fibroblast cells. (FIG. 8E) Effect of adding TSA and/or 5-AZA-C on RNA-mediated alteration of SMN2 splicing.

FIG. 9. Redirection of alternative splicing of mouse APOB by RNA duplexes. Mouse hepatocyte BNL CL.2 (ATCC, TIB-73™) was seeded into 6-well plates. Twenty-four hr later, RNA duplex alone or duplex mixture (final concentration 100 nM) was transfected into cells using Lipofectamine RNAiMAX (Invitrogen) according to the manufacturer's instructions. Total RNA was isolated 24 hr after transfection with Trizol reagent (Invitrogen) and RT-PCR was performed to detect the truncated isoform APOB_{ΔE27}. BPS: branch-point sequence.

FIG. 10. Splicing scheme for Bcl-x_{L} and Bcl-x_{S}.

FIG. 11. Bcl-x siRNA sequences to shift splicing in favor of Bcl-x_{S}. All siRNAs contain one mismatched base at position 10 (in bold) (SEQ ID NOS:42-45). Targeted Bcl-x exon2/intron junction region (SEQ ID NO:46).

FIG. 12. Insulin receptor (IR) A/B splicing scheme and siRNA shifting splicing in favor of IR-B. Targeted intron sequence (SEQ ID NO:47). siRNA sequences (SEQ ID NOS:48-50)

### DETAILED DESCRIPTION OF THE INVENTION

As discussed above, splicing-related diseases present significant health issues. As shown in the data here presented by the inventors, double-stranded RNAs can exploit alternative splicing mechanisms in order to shift splicing and achieve suppression of disease-related alternative spliced products. The use of mismatches in the targeting dsRNAs, as well as chemically-modified bases (in one or both strands) also retain selective inhibitory activity. These and other aspects of the invention are described in detail below.

### I. ALTERNATIVE SPLICING AND SPLICING-RELATED DISEASE DEFECTS

When a pre-mRNA has been transcribed from eukaryotic DNA, it includes several introns and exons. The exons to be retained in the mRNA are determined during the splicing process. The regulation and selection of splice sites are done by trans-acting splicing activator and splicing repressor proteins. The typical eukaryotic nuclear intron has consensus sequences defining important regions. Each intron has GU at its 5′ end. Near the 3′ end there is a branch site. The nucleotide at the branch point is always an A; the consensus around this sequence varies somewhat. In humans the branch consensus is yUnAy. The branch site is followed by a series of pyrimidines, or polypyrimidine tract, then by AG at 3′ end.

Splicing of mRNA is performed by an RNA and protein complex known as the spliceosome, containing snRNPs designated U1, U2, U4, U5, and U6 (U3 is not involved in mRNA splicing). U1 binds to 5′ GU and U2 binds to branch site (A) with the assistance of the U2AF protein factors. The complex at this stage is known as the spliceosome A complex. Formation of the A complex is usually the key step in determining the ends of the intron to be spliced out, and defining the ends of the exon to be retained. The U4, U5, U6 complex binds, and U6 replaces the U1 position. U1 and U4 leave. The remaining complex then performs two transesterification reactions. In the first transesterification, 5′ end of the intron is cleaved from the upstream exon and joined to the branch site A by a 2′,5′-phosphodiester linkage. In the second transesterification, the 3′ end of the intron is cleaved from the downstream exon, and the two exons are joined by a phosphodiester bond. The intron is then released in lariat form and degraded.

Alternative splicing (or differential splicing) is a process by which the exons of the RNA produced by transcription of a gene (a primary gene transcript or pre-mRNA) are reconnected in multiple ways during RNA splicing. The resulting different mRNAs may be translated into different protein isoforms; thus, a single gene may code for multiple proteins.

Alternative splicing occurs as a normal phenomenon in eukaryotes, where it greatly increases the diversity of proteins that can be encoded by the genome; in humans, ˜95% of multiexonic genes are alternatively spliced. There are numerous modes of alternative splicing observed, of which the most common is exon skipping. In this mode, a particular exon may be included in mRNAs under some conditions or in particular tissues, and omitted from the mRNA in others.

The production of alternatively spliced mRNAs is regulated by a system of trans-acting proteins that bind to cis-acting sites on the pre-mRNA itself. Such proteins include splicing activators that promote the usage of a particular splice site, and splicing repressors that reduce the usage of a particular site. Mechanisms of alternative splicing are highly variable, and new examples are constantly being found, particularly through the use of high-throughput techniques. Researchers hope to fully elucidate the regulatory systems involved in splicing, so that alternative splicing products from a given gene under particular conditions could be predicted by a “splicing code.”

Five basic modes of alternative splicing are generally recognized: (a) exon skipping or cassette exon—in this case, an exon may be spliced out of the primary transcript or retained (this is the most common mode in mammalian pre-mRNAs); (b) mutually exclusive exons—one of two exons is retained in mRNAs after splicing, but not both; (c) alternative donor site—an alternative 5′ splice junction (donor site) is used, changing the 3′ boundary of the upstream exon; (d) alternative acceptor site—an alternative 3′ splice junction (acceptor site) is used, changing the 5′ boundary of the downstream exon; and (e) intron retention—a sequence may be spliced out as an intron or simply retained. The latter is distinguished from exon skipping because the retained sequence is not flanked by introns. If the retained intron is in the coding region, the intron must encode amino acids in frame with the neighboring exons, or a stop codon or a shift in the reading frame will cause the protein to be non-functional. This is the rarest mode in mammals.

Changes in the RNA processing machinery may lead to mis-splicing of multiple transcripts, while single-nucleotide alterations in splice sites or cis-acting splicing regulatory sites may lead to differences in splicing of a single gene, and thus in the mRNA produced from a mutant gene's transcripts. A probabilistic analysis indicates that over 60% of human disease-causing mutations affect splicing rather than directly affecting coding sequences.

Abnormally spliced mRNAs are also found in a high proportion of cancerous cells. Until recently, it was unclear whether such aberrant patterns of splicing played a role in causing cancerous growth, or were merely a consequence of cellular abnormalities associated with cancer. It has been shown that there is actually a reduction of alternative splicing in cancerous cells compared to normal ones, and the types of splicing differ; for instance, cancerous cells show higher levels of intron retention than normal cells, but lower levels of exon skipping. Some of the differences in splicing in cancerous cells may result from changes in phosphorylation of trans-acting splicing factors. Others may be produced by changes in the relative amounts of splicing factors produced; for instance, breast cancer cells have been shown to have increased levels of the splicing factor SF2/ASF.

One example of a specific splicing variant associated with cancers is in one of the human DNMT genes. Three DNMT genes encode enzymes that add methyl groups to DNA, a modification that often has regulatory effects. Several abnormally spliced DNMT3B mRNAs are found in tumors and cancer cell lines. Another example is the Ron (MST1R) proto-oncogene. An important property of cancerous cells is their ability to move and invade normal tissue. Production of an abnormally spliced transcript of Ron has been found to be associated with increased levels of the SF2/ASF in breast cancer cells. The abnomal isoform of the Ron protein encoded by this mRNA leads to cell motility. Recent studies point to a key function of chromatin structure and histone modifications in alternative splicing regulation. These insights suggest that epigenetic regulation determines not only what parts of the genome are expressed but also how they are spliced.

A. Duschenne Muscular Dystrophy

Duchenne muscular dystrophy (DMD) is a severe recessive X-linked form of muscular dystrophy characterized by rapid progression of muscle degeneration, eventually leading to loss of ambulation and death. This affliction affects one in 4000 males, making it the most prevalent of muscular dystrophies. In general, only males are affected, though females can be carriers. Females may be afflicted if the father is afflicted and the mother is also a carrier/affected. The disorder is caused by a mutation in the dystrophin gene, located in humans on the X chromosome (Xp21). The dystrophin gene codes for the protein dystrophin, an important structural component within muscle tissue. Dystrophin provides structural stability to the dystroglycan complex (DGC), located on the cell membrane.

Symptoms usually appear in male children before age 5 and may be visible in early infancy. Progressive proximal muscle weakness of the legs and pelvis associated with a loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking but most patients are wheelchair dependent by age 12. Later symptoms may include abnormal bone development that lead to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for patients afflicted with DMD varies from late teens to early to mid 20s. There have been reports of a few DMD patients surviving to the age of 40, but this is extremely rare.

Duchenne muscular dystrophy is caused by a mutation of the dystrophin gene at locus Xp21. Dystrophin is responsible for connecting the cytoskeleton of each muscle fiber to the underlying basal lamina (extracellular matrix) through a protein complex containing many subunits. The absence of dystrophin permits excess calcium to penetrate the sarcolemma (cell membrane). Alterations in these signalling pathways cause water to enter into the mitochondria which then burst. In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue. England et al. (1990) noticed that a patient with mild Beckers' muscular dystrophy was lacking 46% of his coding region for dystrophin. This functional, yet truncated, form of dystrophin gave rise to the notion that shorter dystrophin can still be therapeutically beneficial.

The main symptom of Duchenne muscular dystrophy, a progressive neuromuscular disorder, is muscle weakness associated with muscle wasting with the voluntary muscles being first affected, especially affecting the muscles of the hips, pelvic area, thighs, shoulders, and calf muscles. Muscle weakness also occurs in the arms, neck, and other areas, but not as early as in the lower half of the body. Calves are often enlarged. Symptoms usually appear before age 6 and may appear as early as infancy. The other physical symptoms are:
awkward manner of walking, stepping, or running;
frequent falls;
fatigue;
difficulty with motor skills (running, hopping, jumping);
increased lumbar lordosis, leading to shortening of the hip-flexor muscles;
muscle contactures of achilles tendon and hamstrings;
progressive difficulty walking;
muscle fibre deformities;
pseudohypertrophy (enlarging) of tongue and calf muscles;
higher risk of neurobehavioral disorders (e.g., ADHD, Autistic-Spectrum Disorders), learning disorders (dyslexia), and non-progressive weaknesses in specific cognitive skills (in particular short-term verbal memory);
eventual loss of ability to walk (usually by the age of 12); and
skeletal deformities (including scoliosis in some cases)

The muscle-specific isoform of the dystrophin gene is composed of 79 exons, and DNA testing and analysis can usually identify the specific type of mutation of the exon or exons that are affected. DNA testing confirms the diagnosis in most cases. If DNA testing fails to find the mutation, a muscle biopsy test may be performed. A small sample of muscle tissue is extracted (usually with a scalpel instead of a needle) and a dye is applied that reveals the presence of dystrophin. Complete absence of the protein indicates the condition. Over the past several years DNA tests have been developed that detect more of the many mutations that cause the condition, and muscle biopsy is not required as often to confirm the presence of Duchenne's.

If one or both parents are ‘carriers’ of a particular condition there is a risk that their unborn child will be affected by that condition. ‘Prenatal tests’ are carried out during pregnancy, to try to find out if the fetus (unborn child) is affected. The tests are only available for some neuromuscular disorders. Different types of prenatal tests can be carried out after about 11 weeks of pregnancy. Chorion villus sampling (CVS) can be done at 11-14 weeks, and amniocentesis after 15 weeks, while fetal blood sampling can be done at about 18 weeks. Women and/or couples need to consider carefully which test to have and to discuss this with their genetic counselor. Earlier testing would allow early termination, but it carries a slightly higher risk of miscarriage than later testing (about 2%, as opposed to 0.5%).

There is no known cure for Duchenne muscular dystrophy, although recent stem-cell research is showing promising vectors that may replace damaged muscle tissue. Treatment is generally aimed at controlling the onset of symptoms to maximize the quality of life, and include the following:
Corticosteroids such as prednisolone and deflazacort increase energy and strength and defer severity of some symptoms.
Randomised control trials have shown that beta2-agonists increase muscle strength but do not modify disease progression. Follow-up time for most RCTs on beta2-agonists is only around 12 months and hence results cannot be extrapolated beyond that time frame.
Mild, non-jarring physical activity such as swimming is encouraged. Inactivity (such as bed rest) can worsen the muscle disease.
Physical therapy is helpful to maintain muscle strength, flexibility, and function.
Orthopedic appliances (such as braces and wheelchairs) may improve mobility and the ability for self-care. Form-fitting removable leg braces that hold the ankle in place during sleep can defer the onset of contractures.
Appropriate respiratory support as the disease progresses is important
   A recent report from Goemans et al. (NEJM, on-line Mar. 23, 2011) showed that in a phage 1-2a study, an antisense oligonucleotide could induce the skipping of exon 51 during pre-mRNA splicing to facilitate new dystrophin expression in muscle fiber membranes.

Duchenne muscular dystrophy eventually affects all voluntary muscles and involves the heart and breathing muscles in later stages. The life expectancy typically ranges from the late teens to the mid-30s. Recent advancements in medicine are extending the lives of those afflicted. In rare cases, persons with DMD have been seen to survive into the forties or early fifties, with the use of proper positioning in wheelchairs and beds, ventilator support (via tracheostomy or mouthpiece), airway clearance, and heart medications, if required. Early planning of the required supports for later-life care has shown greater longevity in people living with DMD.

B. Spinal Muscular Atrophy

Spinal Muscular Atrophy (SMA) is a neuromuscular disease characterized by degeneration of motor neurons, resulting in progressive muscular atrophy (wasting away) and weakness. The clinical spectrum of SMA ranges from early infant death to normal adult life with only mild weakness. These patients often require comprehensive medical care involving multiple disciplines, including pediatric pulmonology, pediatric neurology, pediatric orthopedic surgery, Lower Extremity & Spinal Orthosis, pediatric critical care, and physical medicine and rehabilitation; and physical therapy, occupational therapy, respiratory therapy, and clinical nutrition. Genetic counseling is also helpful for the parents and family members. Sensation and the ability to feel are not affected. Intellectual activity is normal and it is often observed that patients with SMA are unusually bright and sociable. The term “juvenile spinal muscular atrophy” refers to Kugelberg-Welander syndrome.

In all of its forms, the primary feature of SMA muscle weakness, accompanied by atrophy of muscle. This is the result of denervation, or loss of the signal to contract, that is transmitted from the spinal cord. This is normally transmitted from motor neurons in the spinal cord to muscle via the motor neuron's axon, but either the motor neuron with its axon, or the axon itself, is lost in all forms of SMA.

The features of SMA are strongly related to its severity and age of onset. SMA caused by mutation of the SMN gene has a wide range, from infancy to adult, fatal to trivial, with different affected individuals manifesting every shade of impairment between these two extremes. Many of the symptoms of SMA relate to secondary complications of muscle weakness, and as such can be at least partially remediated by prospective therapy.

Infantile SMA is the most severe form. Some of the symptoms include:
muscle weakness;
poor muscle tone;
weak cry or cough;
limpness or a tendency to flop;
difficulty sucking or swallowing;
accumulation of secretions in the lungs or throat;
bell-shaped torso, caused by breathing using muscles around the abdominal area;
clenched fists with sweaty hands;
flickering/vibrating of the tongue;
head often tilted to one side, even when lying down;
legs that tend to be weaker than the arms;
legs lying in the “frogs leg” position;
hypotonia, areflexia, and multiple congenital contractures (arthrogryposis) associated with loss of anterior horn cells;
feeding difficulties;
increased susceptibility to respiratory tract infections;
bowel/bladder weakness;
lower-than-normal weight;
developmental milestones, such as lifting the head or sitting up, can't be reached

The most common form of SMA is caused by mutations of the SMN gene, and manifests over a wide range of severity affecting infants through adults. The SMN gene is found on chromosome 5, and the affected SMN gene is called SMN1. Other forms of spinal muscular atrophy are caused by mutation of other genes, some known and others not yet defined. All forms of SMA have in common weakness caused by denervation, that is, muscle weakens because muscle fibers lose the connection from the spinal cord that communicates when to contract.

In order to be diagnosed with Spinal Muscular Atrophy, symptoms need to be present. In most cases, a diagnosis can be made by the SMN gene test, which determines whether there is at least one copy of the SMN1 gene by looking for its unique sequences (that distinguish it from the almost identical SMN2) in exons 7 and 8. In some cases, when the SMN gene test is not possible or does not show any abnormality, other tests such as an EMG electromyography (EMG) or muscle biopsy may be indicated.

Individuals with SMA are living longer and fuller lives with the help of assistive technology such as ventilators, power wheelchairs, and modified access to computers. These mitigate the effects of SMA upon the individuals' daily lives, allowing them to participate in the community like everyone else.

Ventilation is especially important. The course of SMA is directly related to the severity of weakness. Infants with the severe form of SMA frequently succumb to respiratory disease due to weakness of the muscles that support breathing. Children with milder forms of SMA naturally live much longer although they may need extensive medical support, especially those at the more severe end of the spectrum.

Due to molecular biology, there is a better understanding of SMA. Many experimental treatments are being tested, including gene replacement, stem-cell replacement of motor neurons, and most promising therapies intended to increase the expression of the SMN2 gene or increase the percentage of mRNA transcript from SMN2 that is spliced to the full length form. Other potential therapies are directed to drugs that might enhance residual SMN function, or compensate for its loss. Significant progress has been made in preclincial research towards an effective treatment.

Several drugs have been identified in laboratory experiments that hold promise for patients. To evaluate if these drugs benefit SMA patients, clinical trials are needed. In a clinical trial a new medication is tested while the patients are carefully monitored for their safety and for any possible drug effects, positive or negative.

Some drugs under clinical investigation for the treatment of SMA include Butyrates, Valproic acid, Hydroxyurea, Riluzole and Quinazoline495. Other compounds have been identified that increase SMN gene expression or the percentage of full length SMN transcript spliced from SMN2. These compounds are undergoing further pre-clinical development prior to beginning clinical trials.

Presently, treatment for SMA consists of prevention and management of the secondary effect of chronic motor unit loss. Given that much of the mortality is caused by treatable complications, this is important and may be, even in the long run, as important to maintaining overall function as specific treatment of SMN levels.

C. Other Splicing Diseases

Familial Isolated Growth Hormone Deficiency Type II (IGHD II).

Postnatal growth in humans requires secretion of growth hormone (GH) from the anterior pituitary. Familial isolated GH deficiency type II (IGHD II) is a dominantly inherited disorder caused by mutations in the single GH gene (GH-1), in which the main symptom is short stature (Cogan et al., 1994). GH-1 contains five exons and generates a small amount (5%-10%) of alternatively spliced mRNAs (Lecomte et al., 1987). Full-length GH protein is 22 kD, whereas use of an alternative 3′ splice site that removes the first 45 nt of exon 3 and skipping of exon 3 generate 20-kD and 17.5-kD isoforms, respectively. All IGHD II mutations cause increased alternative splicing of exon 3 by disrupting one of three splicing elements: an ISE, an ESE, or the 5′ splice site (Binder et al., 1996; Cogan et al., 1997; Moseley et al., 2002).

Frasier Syndrome.

Inactivation of the Wilms tumor suppressor gene (WT1) is responsible for ˜15% of Wilms tumors, a pediatric cancer of the kidney (Call et al., 1990; Gessler et al., 1990). The human WT1 pre-mRNA undergoes extensive alternative splicing; however, the only alternative splice conserved among vertebrates is the use of two alternative 5′ splice sites for exon 9 separated by 9 nt that encode lysine-threonine-serine (KTS; Miles et al., 1998). The +KTS and −KTS isoforms are expressed at a constant ratio favoring the +KTS isoform in all tissues and developmental stages that express WT1 (Haber et al., 1991). The majority of individuals with FS were found to have mutations that inactivate the downstream 5′ splice site, resulting in a shift to the −KTS isoform (Barbaux et al., 1997; Kohsaka et al., 1999; Melo et al., 2002).

Frontotemporal Dementia and Parkinsonism Linked to Chromosome 17 (FTDP-17).

Aggregation of the microtubule-associated protein tau into neuronal cytoplasmic inclusions is associated with several neuropathological conditions characterized by progressive dementia including Alzheimer's disease, Pick's disease, and frontotemporal dementia and Parkinsonism linked to Chromosome 17 (FTDP-17; Buee et al. 2000). FTDP-17 is an autosomal dominant disorder caused by mutations in the MAPT gene that encodes tau. MAPT mutations fall into two mechanistic classes. One class includes mutations that alter the biochemical properties of the protein. In vitro analysis of these mutant proteins demonstrated either altered ability to modulate microtubule polymerization or enhanced self-aggregation into filaments that resemble neurofibrillary tangles. A second class of disease-causing mutations that affected splicing was revealed by mutations clustered in and around the alternatively spliced exon 10.

Atypical Cystic Fibrosis.

Cystic fibrosis (CF) is an autosomal recessive disorder caused by loss of function of the cystic fibrosis transmembrane conductance regulator (CFTR) gene. CFTR encodes a cAMP-dependent transmembrane chloride channel that is expressed in secretory epithelium. In the USA, more than two-thirds of individuals affected with CF carry the devastating ΔF508 mutation, which causes a failure of the protein to localize to the apical plasma membrane. Fifty percent of affected individuals are homozygous for this allele, resulting in severe pulmonary and pancreatic disease. However, less frequent, “milder” mutations that retain residual CFTR function are responsible for a range of CF-related disorders including late onset or less severe pulmonary disease, male infertility due to congenital bilateral absence of the vas deferens (CBAVD), and chronic idiopathic pancreatitis (Noone and Knowles 2001). Two polymorphisms in the CFTR gene that contribute to atypical CF phenotypes are located at the 3′ end of intron 8 and directly affect splicing of exon 9. Nearly all individuals express a small fraction of CFTR mRNAs that lack exon 9 and express a nonfunctional protein (Delaney et al., 1993; Strong et al., 1993). It is unclear whether this alternative splice serves a purpose.

Cancer.

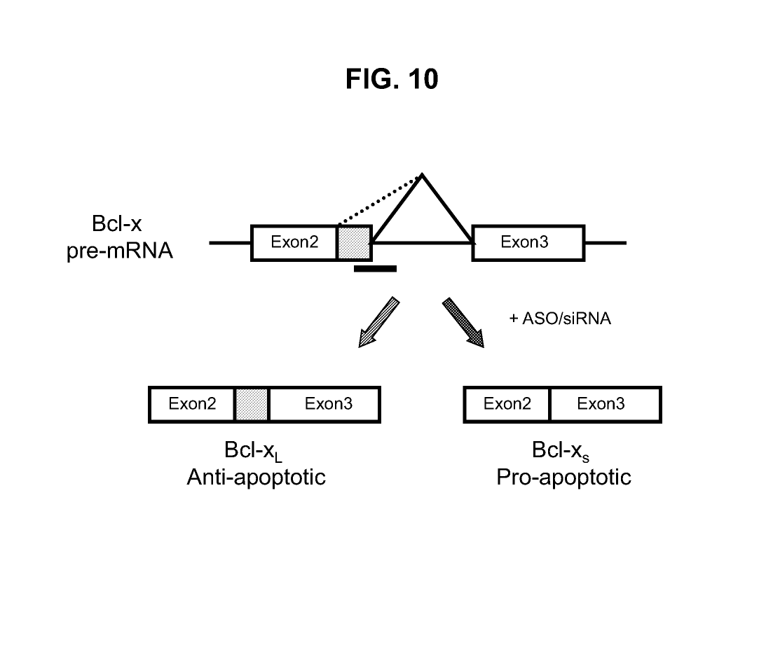

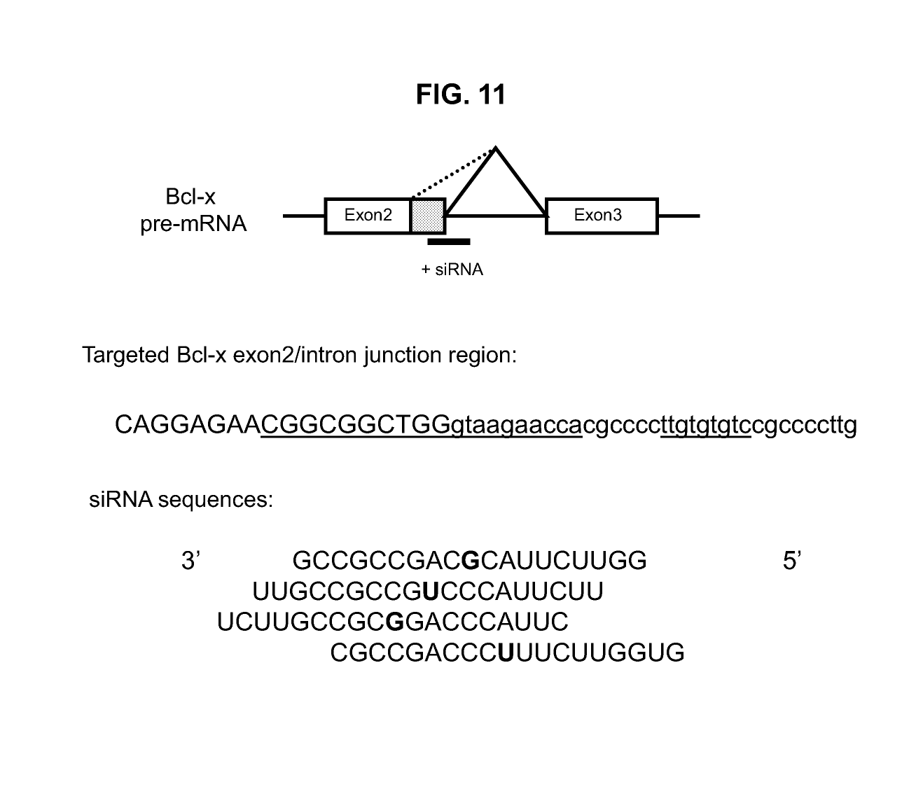

Alternative splicing impacts the development of cancer through a variety of different gene products. For example, alternative splicing of the bcl-x gene generates two isoforms: a longer transcript anti-apoptotic bcl-xL, and a shorter pro-apoptotic bcl-xS. Bcl-xL is overexpressed in many types of cancers and is associated with chemoresistance. Single strand ASO has showed the ability to regulate splicing of Bcl-x and induce apoptosis in cancer cells (FIGS. 10-11).

KLF6, a Kruüppel-like zinc finger transcription factor, plays an important developmental role in hematopoiesis. KLF6 gene encodes a family of proteins generated through alternative splicing. The full length form of the KLF6 gene is a tumor suppressor gene but frequently inactivated by loss of heterozygozity (LOH), somatic mutation, and/or decreased expression in human cancer. KLF6 splice variant 1 (SV1), keeps the majority of the KLF6 N-terminal activation domain but skips all three zinc finger DNA binding domains. KLF6 SV1 acts as a dominant-negative protein that antagonizes full length KLF6. Increased expression of KLF6 SV1 promotes cell proliferation and tumorigenicity. Thus, change of splicing ratio of these two spliceforms will play an important role in anticancer therapy.

| | | Antisense strand |
|---|---|---|
| Candidate gene | Function | (5′-3′) |
| KLF6 | tumor | CCGCUGCGCAGCUUCCCUG |
| (kruppel-like | suppressor | GCUGCGCACGUUCCCUGGC |
| factor 6) | gene | UGCGCACCAUCCCUGGCGA |

Diabetes.

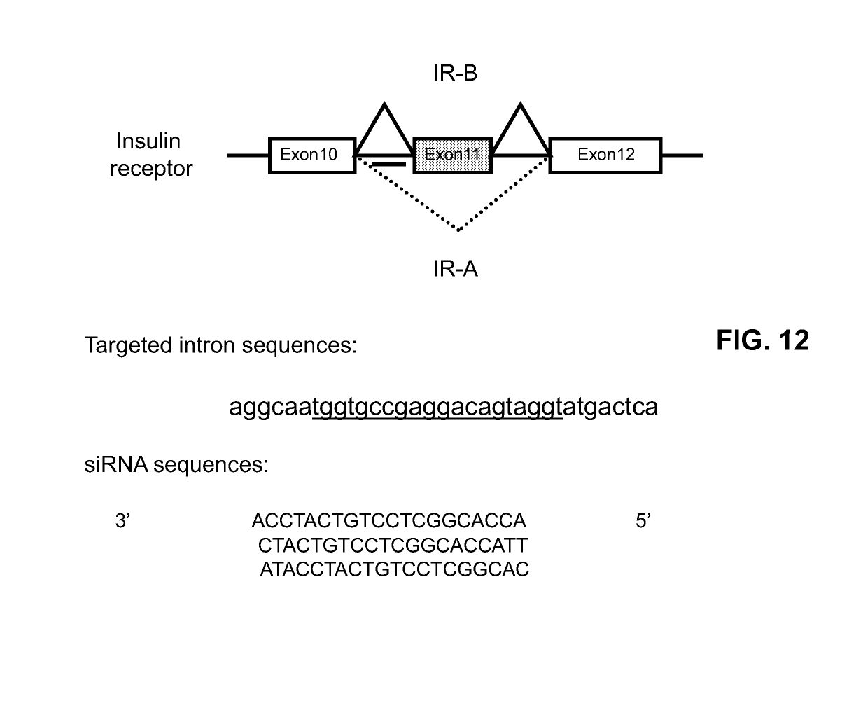

The human insulin receptor (IR) gene is expressed as two tissue-specific isoforms, the shorter variant IR-A and the longer transcript IR-B (with exon 11 inclusion). IR-A is often aberrantly expressed in cancer cells, it may favor cancer resistance to both conventional and targeted therapies by a variety of mechanisms. IR-B is predominantly expressed in adult, well-differentiated tissues, including the liver, where it enhances the metabolic effects of insulin. Dysregulation of IR splicing in insulin target tissues may occur in patients with insulin resistance; however, its role in type 2 diabetes is unclear. Shifting splicing in favor of IR-B may impact insulin resistance (FIG. 12).

### II. OLIGONUCLEOTIDES

The present invention provides double-stranded RNA oligonucleotides that target splice junctions and/or exonic/intronic sequences that flank the same. In general, the RNAs will comprise a segment of about 7-30 ribonucleic acid bases that hybridizes to either a splice junction, an intronic or exonic flanking sequences, or both a splice junction and a flanking region. The length may be 7, 8, 9, 10, 11, 12, 13, 14, 15, 16, 17, 18, 19, 20, 21, 22, 23, 24, 25, 26, 27, 28, 29 or 30 bases in length.

A. Analogs

The double-stranded RNAs may contain non-natural bases and also may contain non-natural backbone linkages.

A locked nucleic acid (LNA), often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. Such oligomers are commercially available. The locked ribose conformation enhances base stacking and backbone pre-organization. This significantly increases the thermal stability (melting temperature) of oligonucleotides (Kaur et al., 2006).

LNA nucleotides are used to increase the sensitivity and specificity of expression in DNA microarrays, FISH probes, real-time PCR probes and other molecular biology techniques based on oligonucleotides. For the in situ detection of miRNA, the use of LNA was as of 2005 the only efficient method. A triplet of LNA nucleotides surrounding a single-base mismatch site maximizes LNA probe specificity unless the probe contains the guanine base of G-T mismatch (You et al., 2006).

Other oligonucleotide modifications can be made to produce the RNAs of the present invention. For example, stability against nuclease degradation has been achieved by introducing a phosphorothioate (P═S) backbone linkage at the 3′ end for exonuclease resistance and 2′ modifications (2′-OMe, 2′-F and related) for endonuclease resistance (WO 2005115481; Li et al., 2005; Choung et al., 2006). A motif having entirely of 2′-O-methyl and 2′-fluoro nucleotides has shown enhanced plasma stability and increased in vitro potency (Allerson et al., 2005). The incorporation of 2′-O-Me and 2′-O-MOE does not have a notable effect on activity (Prakash et al., 2005).

Sequences containing a 4′-thioribose modification have been shown to have a stability 600 times greater than that of natural RNA (Hoshika et al, 2004). Crystal structure studies reveal that 4′-thioriboses adopt conformations very similar to the C3′-endo pucker observed for unmodified sugars in the native duplex (Haeberli et al., 2005). Stretches of 4′-thio-RNA were well tolerated in both the guide and nonguide strands. However, optimization of both the number and the placement of 4′-thioribonucleosides is necessary for maximal potency.

In the boranophosphate linkage, a non-bridging phosphodiester oxygen is replaced by an isoelectronic borane (BH₃—) moiety. Boranophosphate siRNAs have been synthesized by enzymatic routes using T7 RNA polymerase and a boranophosphate ribonucleoside triphosphate in the transcription reaction. Boranophosphate siRNAs are more active than native siRNAs if the center of the guide strand is not modified, and they may be at least ten times more nuclease resistant than unmodified siRNAs (Hall et al., 2004; Hall et al., 2006).

Certain terminal conjugates have been reported to improve or direct cellular uptake. For example, nucleic acid analogs conjugated with cholesterol improve in vitro and in vivo cell permeation in liver cells (Rand et al., 2005). Soutschek et al. (2004) have reported on the use of chemically-stabilized and cholesterol-conjugated siRNAs have markedly improved pharmacological properties in vitro and in vivo. Chemically-stabilized siRNAs with partial phosphorothioate backbone and 2′-O-methyl sugar modifications on the sense and antisense strands (discussed above) showed significantly enhanced resistance towards degradation by exo- and endonucleases in serum and in tissue homogenates, and the conjugation of cholesterol to the 3′ end of the sense strand of an NAA by means of a pyrrolidine linker does not result in a significant loss of gene-silencing activity in cell culture. These studies demonstrates that cholesterol conjugation significantly improves in vivo pharmacological properties of NAAs.

LNA bases may be included in a RNA backbone, but they can also be in a backbone of 2′-O-methyl RNA, 2′-methoxyethyl RNA, or 2′-fluoro RNA. These molecules may utilize either a phosphodiester or phosphorothioate backbone.

U.S. Patent Publication 2008/0015162, incorporated herein by reference, provide additional examples of nucleic acid analogs useful in the present invention. The following excerpts are derived from that document and are exemplary in nature only:

In certain embodiments, oligomeric compounds comprise one or more modified monomers, including 2′-modified sugars, such as nucleosides and nucleotides, with 2′-substituents such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl.

Oligomeric compounds provided herein may comprise one or more monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a bicyclic nucleic acid (BNA). Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

In certain embodiments, oligomeric compounds comprise one or more monomers that is a BNA. In certain such embodiments, BNAs include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA and (E) Oxyamino (4′-CH₂—N(R)—O-2′) BNA.

In certain embodiments, BNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl; substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In one embodiment, each of the bridges of the BNA compounds is, independently, —[C(R₁)(R₂)]ₙ—, —[C(R₁)(R₂)]ₙ—O—, —C(R₁R₂)—N(R₁)—O— or —C(R₁R₂)—O—N(R₁)—. In another embodiment, each of said bridges is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′—wherein each R₁ is, independently, H, a protecting group or C₁-C₁₂ alkyl.

Certain BNA's have been prepared and disclosed in the patent literature as well as in scientific literature (Singh et al., 1998; Koshkin et al., 1998; Wahlestedt et al., 2000; Kumar et al., 1998; WO 94/14226; WO 2005/021570; Singh et al., 1998. Examples of issued US patents and published applications that disclose BNA s include, for example, U.S. Pat. Nos. 7,053,207; 6,268,490; 6,770,748; 6,794,499; 7,034,133; and 6,525,191; and U.S. Patent Publication Nos. 2004/0171570; 2004/0219565; 2004/0014959; 2003/0207841; 2004/0143114; and 2003/0082807.

Also provided herein are BNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., 2001; Braasch et al., 2001; and Orum et al., 2001; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) BNA is used for the bicyclic moiety; in the case of an ethylene group in this position, the term ethyleneoxy (4′-CH₂CH₂—O-2′) BNA is used (Singh et al., 1998; Morita et al., 2003). Methyleneoxy (4′-CH₂—O-2′) BNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., 2000).

An isomer of methyleneoxy (4′-CH₂—O-2′) BNA that has also been discussed is α-L-methyleneoxy (4′-CH₂—O-2′) BNA which has been shown to have superior stability against a 3′-exonuclease. The α-L-methyleneoxy (4′-CH₂—O-2′) BNA's were incorporated into antisense gapmers and chimeras that showed potent antisense activity (Frieden et al., 2003).

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., 1998). BNAs and preparation thereof are also described in WO 98/39352 and WO 99/14226.

Analogs of methyleneoxy (4′-CH₂—O-2′) BNA, phosphorothioate-methyleneoxy (4′-CH₂—O-2′) BNA and 2′-thio-BNAs, have also been prepared (Kumar et al., 1998). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., 1998). In addition, 2′-amino- and 2′-methylamino-BNA's have been prepared and the thermal stability of their duplexes with complementary RNA and DNA strands has been previously reported.

In certain embodiments, the oligomeric compounds comprise one or more high affinity monomers provided that the oligomeric compound does not comprise a nucleotide comprising a 2′-O(CH₂)ₙH, wherein n is one to six. In certain embodiments, the oligomeric compounds including, but no limited to short antisense compounds of the present invention, comprise one or more high affinity monomer provided that the oligomeric compound does not comprise a nucleotide comprising a 2′-OCH₃ or a 2′-O(CH₂)₂OCH₃. In certain embodiments, the oligomeric compounds including, but not limited to short antisense compounds of the present invention, comprise one or more high affinity monomer provided that the oligomeric compound does not comprise a α-L-Methyleneoxy (4′-CH₂—O-2′) BNA. In certain embodiments, the oligomeric compounds including, but no limited to short antisense compounds of the present invention, comprise one or more high affinity monomer provided that the oligomeric compound does not comprise a β-D-Methyleneoxy (4′-CH₂—O-2′) BNA. In certain embodiments, the oligomeric compounds including, but no limited to short antisense compounds of the present invention, comprise one or more high affinity monomer provided that the oligomeric compound does not comprise a α-L-Methyleneoxy (4′-CH₂—O-2′) BNA or a β-D-Methyleneoxy (4′-CH₂—O-2′) BNA.

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleotide backbone of the oligonucleotide. The naturally-occurring linkage or backbone of RNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

In one embodiment, each of the substituted groups, is, independently, mono- or poly-substituted with optionally protected substituent groups independently selected from halogen, oxo, hydroxyl, OJ₁, NJ₁J₂, SJ₁, N₃, OC(═X)J₁, OC(═X)NJ₁J₂, NJ₃C(═X)NJ₁J₂ and CN, wherein each J₁, J₂ and J₃ is, independently, H or C₁-C₆ alkyl, and X is O, S or NJ₁.

In certain such embodiments, each of the substituted groups, is, independently, mono- or poly-substituted with substituent groups independently selected from halogen, oxo, hydroxyl, OJ₁, NJ₁J₂, SJ₁, N₃, OC(═X)J₁, and NJ₃C(═X)NJ₁J₂, wherein each J₁, J₂ and J₃ is, independently, H, C₁-C₆ alkyl, or substituted C₁-C₆ alkyl and X is O or NJ₁.

In one embodiment, each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ₃C(═X)NJ₁J₂ and CN, wherein each J₁, J₂ and J₃ is, independently, H or C₁-C₆ alkyl, and X is O, S or NJ₁.

In one embodiment, each of the substituted groups, is, independently, mono- or poly-substituted with substituent groups independently selected from halogen, oxo, hydroxyl, OJ₁, NJ₁J₂, SJ₁, N₃, OC(═X)J₁, and NJ₃C(═X)NJ₁J₂, wherein each J₁, J₂ and J₃ is, independently, H or C₁-C₆ alkyl, and X is O or NJ₁.

Described herein are linking groups that link monomers (including, but not limited to, modified and unmodified nucleosides and nucleotides) together, thereby forming an oligomeric compound. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligomeric compounds having non-phosphorus linking groups are referred to as oligonucleosides. Modified linkages, compared to natural phosphodiester linkages, can be used to alter, typically increase, nuclease resistance of the oligomeric compound. In certain embodiments, linkages having a chiral atom can be prepared a racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The oligomeric compounds described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids et al. Included in the antisense compounds provided herein are all such possible isomers, as well as their racemic and optically pure forms.

In certain embodiments, provided herein are oligomeric compounds having reactive phosphorus groups useful for forming linkages including for example phosphodiester and phosphorothioate internucleoside linkages. Methods of preparation and/or purification of precursors or oligomeric compounds are not a limitation of the compositions or methods provided herein. Methods for synthesis and purification of oligomeric compounds including DNA, RNA, oligonucleotides, oligonucleosides, and antisense compounds are well known to those skilled in the art.

Generally, oligomeric compounds comprise a plurality of monomeric subunits linked together by linking groups. Nonlimiting examples of oligomeric compounds include primers, probes, antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

In certain embodiments, the present invention provides chimeric oligomeric compounds. In certain such embodiments, chimeric oligomeric compounds are chimeric oligonucleotides. In certain such embodiments, the chimeric oligonucleotides comprise differently modified nucleotides. In certain embodiments, chimeric oligonucleotides are mixed-backbone antisense oligonucleotides. In general, a chimeric oligomeric compound will have modified nucleosides that can be in isolated positions or grouped together in regions that will define a particular motif. Any combination of modifications and/or mimetic groups can comprise a chimeric oligomeric compound as described herein. In certain embodiments, chimeric oligomeric compounds typically comprise at least one region modified so as to confer increased resistance to nuclease degradation, increased cellular uptake, and/or increased binding affinity for the target nucleic acid. In certain embodiments, an additional region of the oligomeric compound may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

B. Design Considerations

The present invention contemplates the production of inhibitory double-stranded RNAs targeting splice junctions or sequences flanking the same. Thus, a primary design consideration is matching the sequence of the target. However, other considerations are important as well. For example, the present inventors have determined that the dsRNAs of the present invention are working in AGO2-mediated fashion. AGO2 is associated with catalytic cleavage of the RNA target and is often referred to as the “catalytic engine” of RNAi. By contrast, AGO1 lacks key catalytic residues and would not be expected to cleave its target.

Knowing that AGO2 is involved suggests the need to incorporate mismatches into the central portion of the dsRNA if one wants to avoid destruction of the target. For example, if the target is within an exon and one wish that wish that exon to be included, but mismatches are important. In contrast, involvement of AGO2 will not affect the exon-excluded mature mRNA, because the exon in that situation would be removed. However, the remaining exon-included RNA will be degraded. If one doesn't want to eliminate the exon-included product (for example, because it retains useful functions), one would want to use a mismatch-containing RNA. Thus, if AGO1 were involved, there would be no need to use mismatch-containing duplexes. AGO2 involvement means that one needs mismatches if one wants to preserve exon-included products.

Thus, another design consideration is the potential placement of 1, 2, or 3 “mismatches” in the double-stranded RNA as compared to the target sequence. The mismatches are generally “centrally located” in the RNA, i.e., not located within the first two or last two bases of the RNA, and the guide strand is targeted in particular. A more restrictive definition of centrally located would be the center 3-4 bases, or in the center base (for an odd number of bases) or one or both of the center bases (for an even number of bases). More particularly, on a nucleic acid of at least 15 residues in length, there should be at least 7 residues flanking each side of the mismatch base, or on a nucleic acid of at least 16 residues in length, there should be at least 7 residues flanking two adjacent mismatched bases. Though any mismatch is useful, of particular interest are purine mismatches, such as introducing an adenosine base into the guide strand.

Another consideration is to avoid multiple changes in the “seed” sequence of the double-stranded RNA, i.e., the first 8 bases. Thus, in a double-stranded RNA of at least 19 bases, there would no or one mismatches in the first 8 bases, and 1-5 mismatches in bases 9-19, or in bases 9 to the 3′-terminus if the molecule is longer than 19 bases. In other words, with respect to multiple mismatches, these can be either in the guide strand, or in both strands, and only one mismatch should occur in the seed region.

### III. TREATMENT OF SPLICING-ASSOCIATED DISEASES

The present invention also involves the treatment of diseases that are, at least in part, caused by defects in/alternative splicing, discussed above. By treatment, it is not necessary that all symptoms of the disease be addressed, or that any degree of “cure” be achieved. Rather, to accomplish a meaningful treatment, all that is required is that one or more symptoms of the disease be ameliorated to some degree, an advantageous effect be provided in combination with another therapy, or that the disease progression be slowed.

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals. One will generally desire to employ appropriate salts, buffers, and lipids to render delivery of the oligonucleotides to allow for uptake by target cells. Such methods an compositions are well known in the art, for example, as disclosed in U.S. Pat. Nos. 6,747,014 and 6,753,423. Compositions of the present invention comprise an effective amount of the oligonucleotide to cells, dissolved or dispersed in a pharmaceutically acceptable carrier or medium.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents, liposomes, cationic lipid formulations, microbubble nanoparticles, and the like. The use of such media and agents for pharmaceutically active substances is well-known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral or parenteral routes, including intracranial (including intraparenchymal and intraventricular), intrathecal, epidural, intravenous, intraarterial, intramuscular, intraperitoneal, subcutaneous, transdermal, airway (aerosol), nasal, rectal, vaginal and topical (including buccal and sublingual) administration. Compositions would normally be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, lipids, nanoparticles, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the NAAs of the present invention may be incorporated with excipients. The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intraarterial, intramuscular, subcutaneous, intraperitoneal, intrathecal, epidural and intracranial (including intraparenchymal and intraventricular) administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Lipid vehicles encompass micelles, microemulsions, macroemulsions, liposomes, and similar carriers. The term micelles refers to colloidal aggregates of amphipathic (surfactant) molecules that are formed at a well-defined concentration known as the critical micelle concentration. Micelles are oriented with the nonpolar portions at the interior and the polar portions at the exterior surface, exposed to water. The typical number of aggregated molecules in a micelle (aggregation number) is 50 to 100. Microemulsions are essentially swollen micelles, although not all micellar solutions can be swollen to form microemulsions. Microemulsions are thermodynamically stable, are formed spontaneously, and contain particles that are extremely small. Droplet diameters in microemulsions typically range from 10-100 nm. In contrast, the term macroemulsions refers to droplets with diameters greater than 100 nm. Liposomes are closed lipid vesicles comprising lipid bilayers that encircle aqueous interiors. Liposomes typically have diameters of 25 nm to 1 μm (see, e.g., Shah, 1998; Janoff, 1999).

In one embodiment of a liposome formulation, the principal lipid of the vehicle may be phosphatidylcholine. Other useful lipids include various natural (e.g., tissue derived L-α-phosphatidyl: egg yolk, heart, brain, liver, soybean) and/or synthetic (e.g., saturated and unsaturated 1,2-diacyl-SN-glycero-3-phosphocholines, 1-acyl-2-acyl-SN-glycero-3-phosphocholines, 1,2-diheptanoyl-SN-glycero-3-phosphocholine) derivatives of the same. Such lipids can be used alone, or in combination with a secondary lipid. Such secondary helper lipids may be non-ionic or uncharged at physiological pH, including non-ionic lipids such as cholesterol and DOPE (1,2-dioleolylglyceryl phosphatidylethanolamine). The molar ratio of a phospholipid to helper lipid can range from about 3:1 to about 1:1, from about 1.5:1 to about 1:1, and about 1:1.

Another specific lipid formulation comprises the SNALP formulation, containing the lipids 3-N-[(ω methoxypoly(ethylene glycol)₂₀₀₀)carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar % ratio. See Zimmerman et al. (2006).

A liposome is, in simplest form, composed of two lipid layers. The lipid layer may be a monolayer, or may be multilamellar and include multiple layers. Constituents of the liposome may include, for example, phosphatidylcholine, cholesterol, phosphatidylethanolamine, etc. Phosphatidic acid, which imparts an electric charge, may also be added. Exemplary amounts of these constituents used for the production of the liposome include, for instance, 0.3 to 1 mol, 0.4 to 0.6 mol of cholesterol; 0.01 to 0.2 mol, 0.02 to 0.1 mol of phosphatidylethanolamine; 0.0 to 0.4 mol, or 0-0.15 mol of phosphatidic acid per 1 mol of phosphatidylcholine.

Liposomes can be constructed by well-known techniques (see, e.g., Gregoriadis (1993). Lipids are typically dissolved in chloroform and spread in a thin film over the surface of a tube or flask by rotary evaporation. If liposomes comprised of a mixture of lipids are desired, the individual components are mixed in the original chloroform solution. After the organic solvent has been eliminated, a phase consisting of water optionally containing buffer and/or electrolyte is added and the vessel agitated to suspend the lipid. Optionally, the suspension is then subjected to ultrasound, either in an ultrasonic bath or with a probe sonicator, until the particles are reduced in size and the suspension is of the desired clarity. For transfection, the aqueous phase is typically distilled water and the suspension is sonicated until nearly clear, which requires several minutes depending upon conditions, kind, and quality of the sonicator. Commonly, lipid concentrations are 1 mg/ml of aqueous phase, but could be higher or lower by about a factor of ten.

Lipids, from which the solvents have been removed, can be emulsified by the use of a homogenizer, lyophilized, and melted to obtain multilamellar liposomes. Alternatively, unilamellar liposomes can be produced by the reverse phase evaporation method (Szoka and Papahadjopoulos, 1978). Unilamellar vesicles can also be prepared by sonication or extrusion. Sonication is generally performed with a bath-type sonifier, such as a Branson tip sonifier (G. Heinemann Ultrashall and Labortechnik, Schwabisch Gmund, Germany) at a controlled temperature as determined by the melting point of the lipid. Extrusion may be carried out by biomembrane extruders, such as the Lipex Biomembrane Extruder (Northern Lipids Inc, Vancouver, British Columbia, Canada). Defined pore size in the extrusion filters may generate unilamellar liposomal vesicles of specific sizes. The liposomes can also be formed by extrusion through an asymmetric ceramic filter, such as a Ceraflow Microfilter (commercially available from the Norton Company, Worcester, Mass.).

Following liposome preparation, the liposomes that have not been sized during formation may be sized by extrusion to achieve a desired size range and relatively narrow distribution of liposome sizes. A size range of about 0.2-0.4 microns will allow the liposome suspension to be sterilized by filtration through a conventional filter (e.g., a 0.22 micron filter). The filter sterilization method can be carried out on a high throughput basis.

Several techniques are available for sizing liposomes to a desired size, including, ultrasonication, high-speed homogenization, and pressure filtration (Hope et al., 1985; U.S. Pat. Nos. 4,529,561 and 4,737,323). Sonicating a liposome suspension either by bath or probe sonication produces a progressive size reduction down to small unilamellar vesicles less than about 0.05 microns in size. Multilamellar vesicles can be recirculated through a standard emulsion homogenizer until selected liposome sizes, typically between about 0.1 and 0.5 microns. The size of the liposomal vesicles may be determined by quasi-elastic light scattering (QELS) (see Bloomfield, 1981). Average liposome diameter may be reduced by sonication of formed liposomes. Intermittent sonication cycles may be alternated with QELS assessment to guide efficient liposome synthesis.

Liposomes can be extruded through a small-pore polycarbonate membrane or an asymmetric ceramic membrane to yield a well-defined size distribution. Typically, a suspension is cycled through the membrane one or more times until the desired liposome size distribution is achieved. The liposomes may be extruded through successively smaller-pore membranes, to achieve a gradual reduction in liposome size. For use in the present invention, liposomes have a size of about 0.05 microns to about 0.5 microns, or having a size of about 0.05 to about 0.2 microns.

It is common in many fields of medicine to treat a disease with multiple therapeutic modalities, often called “combination therapies.” Therapies would be provided in a combined amount effective to achieve a reduction in one or more disease parameter. This process may involve contacting subjects with the both agents/therapies at the same time, e.g., using a single composition or pharmacological formulation that includes both agents, or by contacting the subject with two distinct compositions or formulations, at the same time, wherein one composition includes the double-stranded RNAs of the present invention and the other includes the other agent.

Alternatively, the double-stranded RNA therapy may precede or follow the other treatment by intervals ranging from minutes to weeks. One would generally ensure that a significant period of time did not expire between the time of each delivery, such that the therapies would still be able to exert an advantageously combined effect on the subject. In such instances, it is contemplated that one would contact the subject with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the double-stranded RNA therapy or the other therapy will be desired. Various combinations may be employed, where the double-stranded RNAis “A,” and the other therapy is “B,” as exemplified below:

A/B/A B/A/B B/B/A A/A/B B/A/A A/B/B B/B/B/A
B/B/A/B
A/A/B/B A/B/A/B A/B/B/A B/B/A/A B/A/B/A
B/A/A/B B/B/B/A
A/A/A/B B/A/A/A A/B/A/A A/A/B/A A/B/B/B
B/A/B/B B/B/A/B

Other combinations are contemplated. Agents or factors suitable for use in a combined therapy include those described above for the various polyglutamine repeat diseases.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating polyglutamine diseases.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Oligonucleotides and Small Molecule Epigenetic Modulators.

Unless otherwise noted, duplex RNAs were purchased from Integrated DNA Technologies (IDT, Coralville, Iowa). Duplex RNAs complementary to Ago1 or Ago2 mRNA were provided by Dharmacon. 2′-O-methyl RNA was obtained from Sigma. Trichostatin A (TSA) and 5-aza-2′-deoxycytidine (5-Aza-dC) were obtained from Sigma. All conclusions derive from multiple independent experiments.

Cell Culture.

HeLa pLuc/705 cells were provided from Dr. Ryszard Kole (Univ. of North Carolina) and cultured at 37° C. and 5% CO₂ in Dulbecco's Modified Eagle's Medium (DMEM) (Sigma, D5796) supplemented with 10% heat inactivated fetal bovine serum (Sigma), 1% Sodium Pyruvate (Sigma) and 0.5% MEM nonessential amino acids (Sigma). Patient-derived SMA type I homozygous fibroblast cell (GM03813; Coriell Cell Repositories, Camden, N.J., United States) were cultured in Minimum Essential Medium Eagle (MEM; Sigma, M4655) supplemented with 10% (v/v) fetal bovine serum and 1% MEM nonessential amino acids (Sigma, M7145).

Transfections.

Hela pLuc/705 cells were plated 24 h in advance so that their density on the day of transfection was ˜90%. For GM03813 fibroblast transfection, cells were plated at a density of ˜1.2×10⁵ per well of a 6-well plate 24 h before transfection. Cells were transfected with siRNAs using RNAiMAX (Invitrogen) (Yue et al., 2010; Chu et al., 2010). Unless indicated otherwise, total RNA was isolated 24 h after transfection with Trizol (Invitrogen) for RT-PCR or luciferase assay.

Luciferase Assay.

Cells were washed twice with 1×PBS and lysed with Passasive Lysis Buffer (Promega, Madison, Wis.). Total protein concentrations were determined by the bicinchoninic acid (BCA assay). Luciferase activity was measured on a Synergy 2 Multi-Mode Microplate Reader (BioTek, Winooski, Vt.) by mixing 50 L of cell lysate with 100 L of Luciferase Assay System substrate (Promega). Luciferase expression was calculated as relative luminescence units (RLU) per microgram protein and shown as fold increase in luminescence compared with negative control. All experiments were performed in multiple independent transfections. Error bars are standard deviation.

RT-PCR.

Total RNA was extracted and treated with DNase I (Worthington Biochemical) at 25° C. for 10 min to generate cDNA. Reverse transcription was performed using High Capacity Reverse Transcription Kit (Applied Biosystems) according to the manufacturer's protocol. Generally, 2.0 μg of total RNA was used per 20 μL of reaction mixture. PCR was performed on a 7500 real-time PCR system (Applied Biosystems) using iTaq SYBR Green Supermix (BioRad) using the following primers for Hela cell: Luci forward primer 5′-TTGATATGTGGATTTCGAGTCGTC-3′ (SEQ ID NO:7) and Luci reverse primer 5′-TGTCAATCAGAGTGCTTTTGGCG-3′ (SEQ ID NO:7). PCR for SMA cell used forward primer: 5′-CCTCCCATATGTCCAGATTCTCTTGATG-3′ (SEQ ID NO:8) and reverse primer: 5′-CAGATGGTTTTTCAAAATAGAGTCC-3′ (SEQ ID NO:9). The PCR products were separated on a 2% agarose gel and visualized on an AlphaImager. The bands were quantified using ImageJ software (Rasband and Image, U. S. National Institutes of Health, Bethesda, Md., USA, rsb.info.nih.gov/ij/, 1997-2007). Inclusion of exon 7 was calculated as a percentage of the total amount of spliced mRNA, i.e., included mRNA*100/(included mRNA+skipped mRNA). The increment of aberrant mRNA was calculated as a-fold mRNA level above a control sample.

RNA Immunoprecipitation (RIP).

Hela Luc/705 cells were grown in 150 cm² dishes and transfected with duplex RNAs. Cells (˜4×10⁷) were harvested 24 h after transfection and nuclear fraction was isolated. A nuclear lysis buffer [150 mM KCl, 20 mM Tris-HCl 7.4, 3 mM MgCl₂, 0.5% NP-40, 1× Roche protease inhibitors cocktail, RNAse·in (50 U/mL final)] was added to the nuclei (note: no formaldehyde cross-linking is used in this protocol) (Chu et al., 2010; Liu et al., 2005). The mixture was left on ice for 10 min. After vigorous vortexing and pipetting, nuclei were freeze-thawed three times in liquid nitrogen and a 22° C. water bath. Insoluble material was removed by centrifugation at maximum speed for 15 min at 4° C. Nuclear extracts were quickly frozen in liquid nitrogen and stored at −80° C. 60 μL Protein A/G agarose Plus was washed with phosphate-buffered saline (1×PBS, pH 7.4) and incubated with 5 μg of anti-AGO1(4B8, SAB4200084, Sigma), anti-AGO2 (4G8, 011-22033, Wako) antibody in 0.5 mL at 4° C. with gentle agitation for 2 hr. After one wash with 1×PBS and one wash with nuclear lysis buffer, beads were incubated with nuclear cell lysate under constant rotation for 3 h at 4° C. After washing with nuclear lysis buffer (3×), the beads were then treated with elution buffer (1% SDS, 0.1M NaHCO₃ and RNase inhibitor). Following proteinase K treatment, RNA extraction and precipitation, samples were treated with recombinant DNase I and reverse transcription was performed. Corresponding cDNA was amplified using reverse primer complementary to Luc/705 pre-mRNA (5′-AAAACGATCCTGAGACTTCCACACTGATG-3′ (SEQ ID NO:10) with Luci forward primer and GAPDH mRNA (Applied Biosystems).

Results were normalized by measuring two parameters simultaneously. (1) The binding of Luc/705 pre-mRNA to IgG, to anti-Ago1 and to anti-Ago2 antibodies as an indicator of Fold enrichment of Luc/705 pre-mRNA in Ago1 or Ago2 IP relative to IgG IP. (2) The binding of GAPDH mRNA (in both Ago1 or Ago2 IP and IgG IP) to the above antibodies as an indicator of a housekeeping control for background binding.

Chromatin Immunoprecipitation (ChIP) Assay.

ChIP assays were performed as described (Yue et al., 2010). Anti-trimethyl-histone H3 (Lys27) and anti-dimethyl-histone H3 (Lys9) antibodies were supplied by Millipore.

### Example 2

### Results

Efforts to develop chemical agents to redirect splicing have focused on single-stranded oligomers including PNAs (Sazani et al., 2002), LNAs (Guterstam et al., 2008), morpholino oligomers (Alter et al., 2006), and 2′-modified oligonucleotides (Dominski and Kole, 1993; Mann et al., 2001). Systemic administration of mopholino oligomers to canine models of DMD redirects splicing in muscles and partially restores mobility (Yakota et al., 2009). Phase I clinical trials demonstrated partial restoration of dystrophin expression (van Deutekom et al., 2007; Kinali et al., 2009).

Short interfering RNAs (siRNAs) offer another strategy for recognizing mRNA and are in clinical trials (Watts and Corey, 2010). Normally, siRNAs bind argonaute 2 (AGO2) (Liu et al., 2004), recognize mRNA in the cytoplasm, guide cleavage of the RNA target by AGO2, and inhibit gene expression. While cleavage of an mRNA target is desirable for inhibition of gene expression, destruction of mRNA would be incompatible with redirecting splicing. The inventors have observed, however, that during transcriptional silencing or activation the AGO2 protein can mediate recognition of RNA targets in the nucleus by duplex RNAs without causing cleavage (Yue et al., 2010; Chu et al., 2010). This observation led them to hypothesize that small RNAs might also have the ability to alter another nuclear event—splicing.

β-globin.

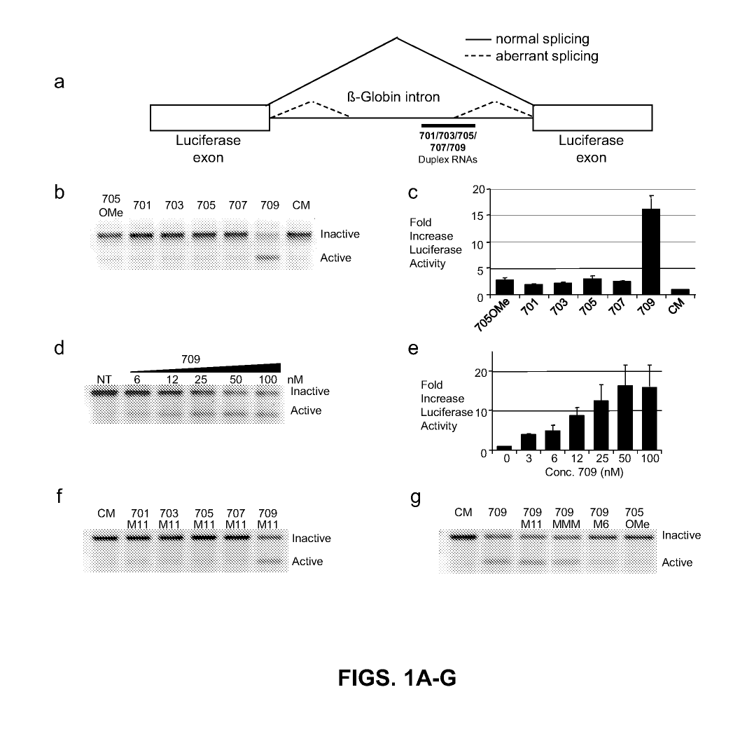

To examine the effect of duplex RNAs on splicing, the inventors used an engineered HeLa cell line (HeLa pLuc705) that expresses a chromosomally-encoded luciferase gene interrupted by intron 2 from β-globin mRNA19 (FIG. 1A). The β-globin intron has been mutated to introduce a new splice site that results in retention of a fragment of the intron and production of truncated luciferase protein. Antisense oligonucleotides complementary to the introduced splice site redirect expression towards normal length luciferase protein. HeLa pLuc705 cells are advantageous for splicing studies because changes I splicing can be monitored at the RNA level by semi-quantitative PCR or at the protein level by luciferase activity.

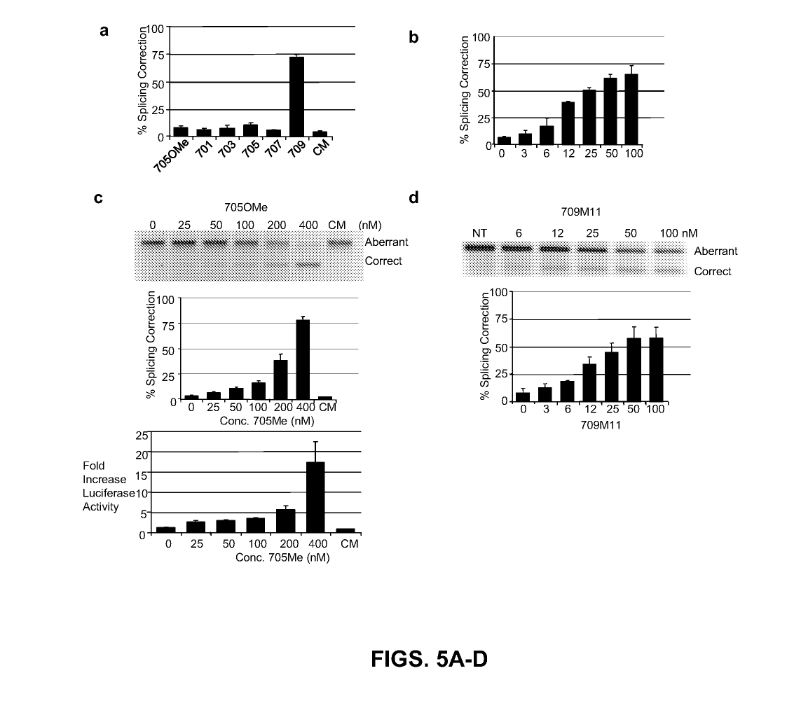

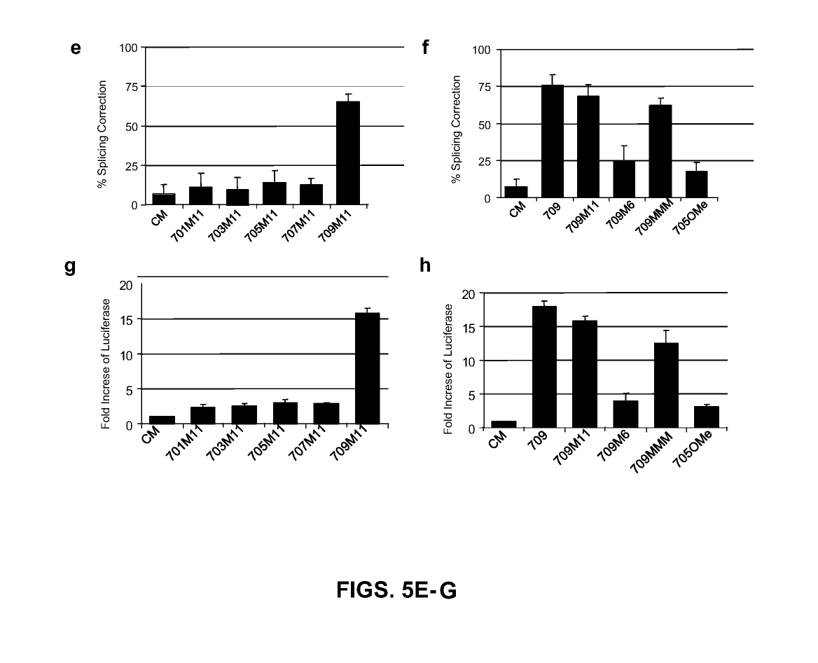

The inventors designed several fully complementary duplex RNAs complementary to sequences near the introduced splice site within the β-globin-derived intron (Table 1). Duplex 709 was a potent activator of luciferase expression while duplex RNAs targeting nearby sites were less active (FIGS. 1B-C, FIG. 5A). Half maximal activity was achieved by addition of 10 nM duplex while previously characterized (Kang et al., 1998) antisense oligomer 705OMe required greater than 200 nM (FIGS. 1D-E, FIG. 5C).

Some sequences that effect splicing are within exons. Duplex RNAs that target these would have the potential to act like standard siRNAs and cause cleavage of mature mRNA containing the exonic target site. It is possible to disrupt cleavage by introducing mismatched bases within the central region (bases 10 or 11) (Du et al., 2005; Wang et al., 2008) of the duplex RNA, and possibly avoid transcript destruction by splice-correcting duplexes that target exons.

To test if mismatch-containing RNAs could be active splice modulators, the inventors tested several different duplexes targeted to sequences near the aberrant splice site. Similar to the inventors' results with fully complementary duplex RNA (709F), only duplex 709M11 was an efficient silencing agent (FIG. 1F). 709M11 yielded a maximal efficiency of 50% with half maximal activity at 10 nM (FIG. 5D) and silencing could be achieved with three centrally located mismatches within a duplex RNA (709MMM) (FIG. 1G, FIGS. 5E-F). Duplex RNAs containing multiple mismatches are similar in structure to miRNAs and their ability to alter splicing suggests that some miRNAs might be capable of splice correction. Recognition of RNA substrates is highly dependent on binding of the seed sequence, bases 2-8 of the duplex RNA. Just one mismatch within the seed sequence (709M6) prevented splice correction (FIG. 1G, FIGS. 5G-H).

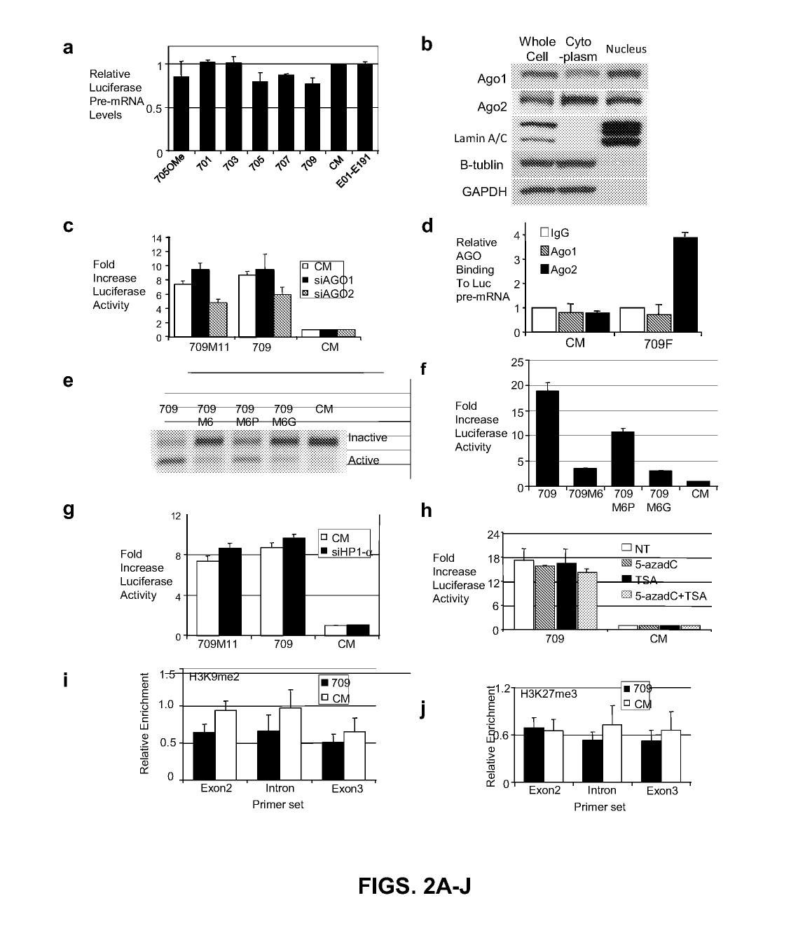

Recognition of mRNA by fully complementary duplex RNAs in the cytoplasm is associated with cleavage of the target RNA. To investigate the mechanism of RNA-mediated splice correction, the inventors checked whether the duplex RNAs used in this experiment affect RNA levels. Several lines of evidence suggest that splice-correcting RNAs can target introns without reducing RNA levels: 1) semi-quantitative PCR showed that levels of luciferase RNA remain relatively constant (FIGS. 1A-G); 2) measurement of luciferase activity indicate that production of luciferase protein was being increased rather than reduced (FIGS. 1A-G, FIGS. 5A-H) and; 3) quantitative PCR reveals no significant change in transcript levels (FIG. 2A).

AGO proteins are critical components of the cellular machinery for recognizing small RNAs and the inventors hypothesized that they might also be involved in RNA-mediated control of splicing. There are four AGO proteins in human cells (AGO1-4). AGO2 is responsible for post-transcriptional gene silencing (Liu et al., 2004) but AGO1 has been reported to be involved in altering splicing through recognition of noncoding RNA (Allo et al., 2009). AGO2 is found in cytoplasmic p-bodies (Sen and Blau, 2005; Liu et al., 2005), but has also been reported to be in the nucleus (Robb et al., 2005). The inventors identified both AGO1 and AGO2 within purified nuclei from HeLa pLuc/705 cells (FIG. 2B).

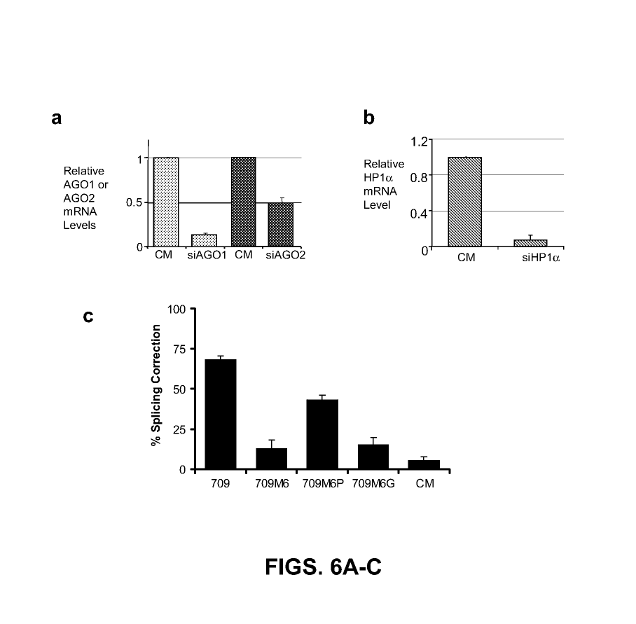

To test which AGO variant might be responsible for the splice correction, the inventors used anti-AGO1 or anti-AGO2 siRNAs to deplete cellular AGO1 or AGO2 (FIG. 6A). Lowering levels of AGO2, but not AGO1, blocked splice correction and production of active luciferase (FIG. 2C). They then used RNA immunoprecipitation (RIP) to examine recruitment of AGO1 and AGO2 to the luciferase transcript upon addition of duplex 709. AGO2, but not AGO1, was recruited to luciferase pre-mRNA (FIG. 2D). The primers for RIP are on either side of the target site for RNA recognition and would not amplify product if the target site were cleaved, providing additional evidence that recognition of RNA targets in the nucleus is not always accompanied by cleavage of mRNA.

Allo et al. (2009) has reported that duplex RNAs can induce inclusion of exons in an AGO1-dependent manner through complementarity to endogenous antisense transcripts or pre-RNA, inducing changes in chromatin structure, and affecting elongation of pre-RNA by RNA polymerase. The mechanism the present inventors observe is fundamentally different. These data suggest a critical role for AGO2 rather than AGO1 (FIGS. 2C-D). RNA 709M6P containing a seed-sequence mismatch relative to a putative antisense noncoding transcript retained activity, whereas RNA 709M6G at position 6 of the strand complementary to the mRNA lost the ability to correct splicing (FIGS. 2E-F), suggesting that mRNA rather than a noncoding transcript was the exclusive target for these splice-correcting RNAs. In contrast to the previous report, inhibition of HP1a expression (FIG. 2G, FIG. 6B) or synthetic epigenetic regulatory agents 5-aza cytidine (5-AZA-C or trichostatin A (TSA) (FIG. 2H) did not affect splice correction. The inventors observed no alteration of histone marks H3K9me2 (FIG. 2I) or H3K27me3 (FIG. 2J).

SMA.

To further characterize RNA-mediated splice correction, the inventors examined a gene containing both exonic and intronic target sequences. Spinal muscular atrophy (SMA) is an inherited neurodegenerative disorder caused by loss or mutation of the survival motor neuron 1 (SMN1) gene (Lorson et al., 2010). A second gene, SMN2, is closely related to SMN1 but its active spliceform is not efficiently expressed. SMA is due to homozygous mutations or deletions in the gene Survival of Motor Neuron 1 (SMN1). SMN2 is paralogous gene of SMN1 and can partially compensate for SMN1 loss of function. SMN1 and SMN2 genes differ in only a few nucleotides and code for identical amino acid sequences. The most relevant nucleotide change is a C→T transition at position 6 in exon 7 that switches splicing to exclude exon 7. Oligonucleotides that alter SMN2 splicing can enhance production of full length SMN2 protein and increase SMN2 protein to therapeutically useful levels that compensate for loss of SMN1 (Burghes and McGovern, 2010; Hua et al., 2010).

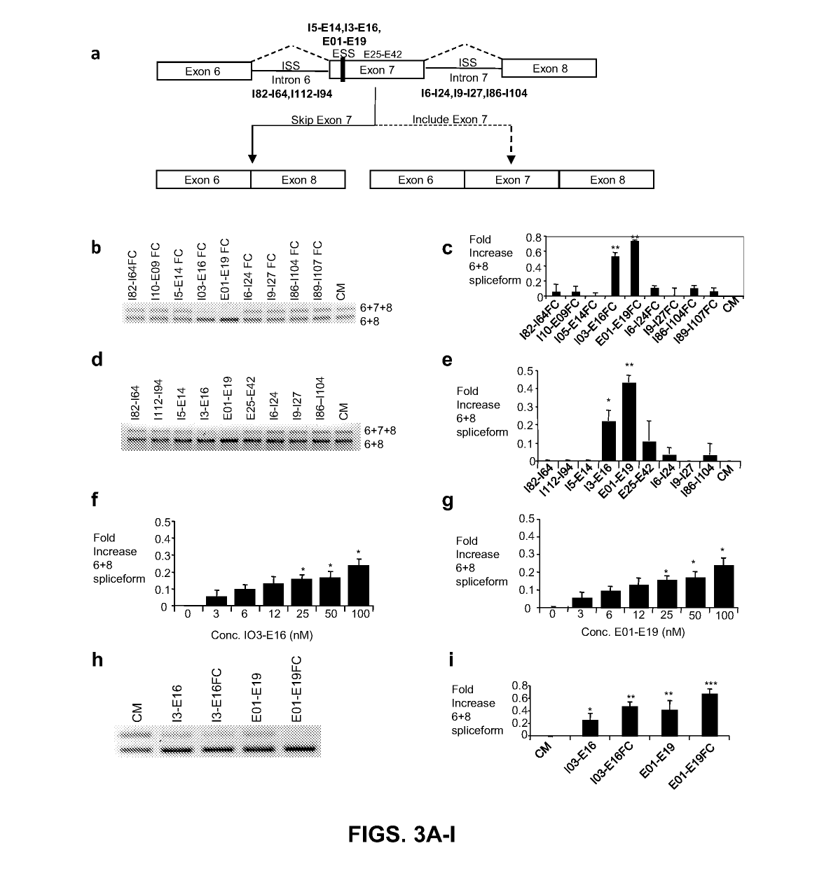

SMN2 can be spliced so as to directly join exons 6 and 8 or to produce a protein that contains exons 6, 7, and 8 (FIG. 3A). The inventors designed duplex RNAs to target sequences near intronic splice silencers (ISSs) or an exonic splice silencer (ESS). Duplexes I03-E16FC and E01-E19FC (numbering is relative to the intron exon junction and FC denotes full complementarity) targeted near the intron 6/exon 7 junction, ESS, and the key C-T mutation increased exclusion of exon 7 (Table 2, FIGS. 3B-C).

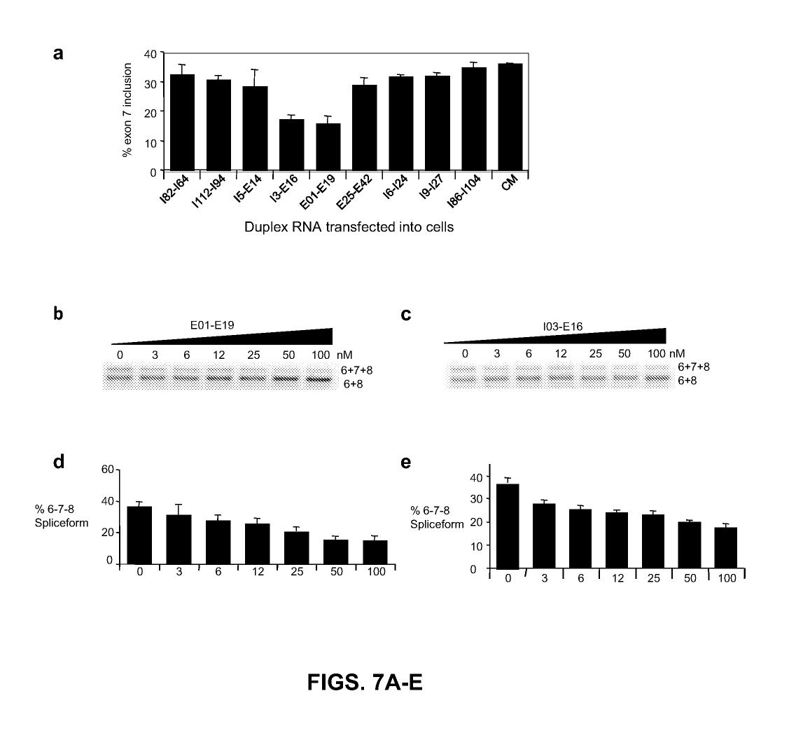

Duplex RNAs complementary to exons can induce cleavage of mature mRNA in the cytoplasm. To avoid cleaving the spliceform that retains exon 7, the inventors introduced mismatches at positions known to disrupt catalysis by AGO2. Mismatch-containing duplexes I3-E16 and E01-E19 produced the most efficient production of the 6+8 spliceform (FIGS. 3D-E) and reduction of exon 7 inclusion (FIG. 7A). Exclusion of exon 7 was dose dependent (FIGS. 3F-G; FIGS. 7B-G). Direct comparison of fully complementary and mismatch-containing duplexes revealed a greater reduction of the 6+7+8 spliceform upon addition of the fully complementary RNA, consistent with cleavage in the cytoplasm (FIGS. 3H-I).

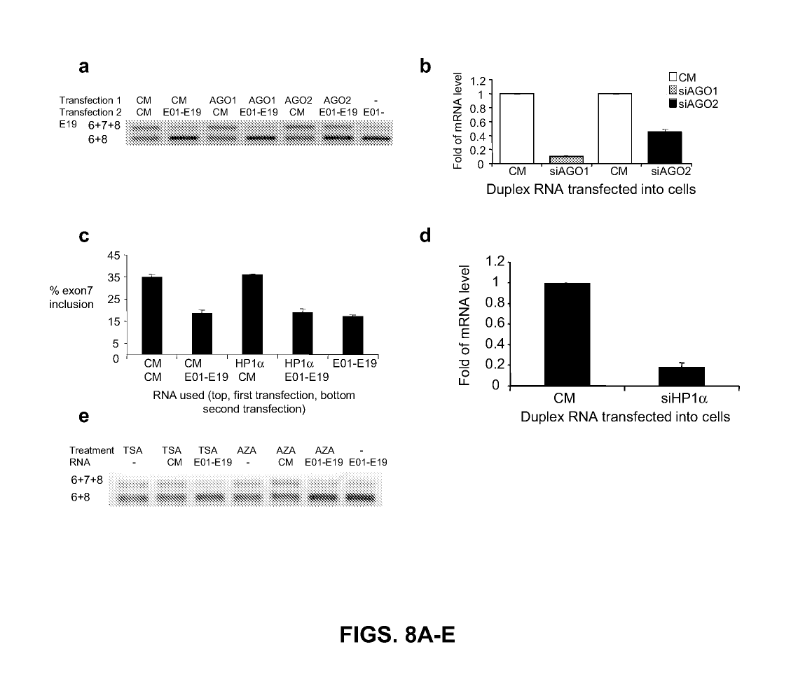

Mismatched duplexes can be used if it is necessary to preserve spliceforms that retain targeted exons, while fully complementary duplexes can be used if maximal biasing towards exon-excluded spliceforms are required. The action of duplex RNAs was reversed by use of siRNAs to reduce AGO2 expression while reduction of AGO1 expression did not affect splicing (FIGS. 8A-B). Inhibition of HP1a, treatment with TSA or 5-AZA-C did not affect expression also had no effect on RNA-mediated alteration of splicing (FIGS. 8C-E).

Dystrophin.

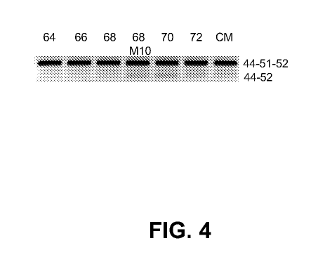

The inventors also examined RNAs complementary to dystrophin pre-mRNA to test their effects on splicing of dystrophin (FIG. 4, Table 3). They targeted sequences within exon 51 because deletion of exon 51 in some patients removes a premature stop codon and can produce partially active dystrophin (Lu et al., 2010). Two RNAs, 68M10 and 70, successfully induced exon exclusion and production of the truncated dystrophin characteristic of the more mild Becker's muscular dystrophy (FIG. 4). This dystrophin data, together with altered splicing of engineering luciferase and SM2, provide three examples of small-RNA mediated exon exclusion. In all cases, only a handful of RNAs were tested prior to identification of active agents, suggesting that the phenomenon is general and easily achievable.

Rigo and colleagues (submitted) have reported that single-stranded 2′-fluoro antisense oligonucleotides can bind interleukin enhancer binding factors 2 and 3 (ILF2/3), recruit the proteins to pre-mRNAs, and alter splicing through a mechanism that differs from analogous antisense oligonucleotides that lack the ability to bind proteins. This protein recruitment has important physiologic consequences, because oligonucleotides with exactly the same sequence were shown to cause opposite effects on spliceform production depending on whether 2′-F was present.

These data complement these studies by showing that native RNAs and endogenous argonaute protein can also partner to redirect splicing. The similar effects upon recruitment of ILF2/3 by 2′-F oligonucleotides or AGO2 by RNA, protein:nucleic acid combinations that differ substantially in their biochemical properties, suggests a simple mechanism of action that redirects splicing rather than a more complex mechanism that requires induction of histone modifications, alteration of chromosomal DNA, and effects on gene transcription. These two examples of nucleic acid-directed protein recruitment expand the range of strategies available for controlling protein expression.

These findings demonstrate that splicing can be modulated by duplex RNAs that recognize mRNA. These results have several implications. First, small RNAs can function in conjunction with nuclear AGO2 to recognize pre-mRNA transcripts and alter splicing, which finding expands the range of RNA-mediated control of gene expression. Second, regulation of splicing is robust, easily reproducible and observed after testing only a handful of duplexes. Small miRNA-like mismatch-containing duplexes also alter splicing. miRNAs exist in the nucleus (Liao et al., 2010) and these data suggest that miRNAs have the potential to modulate splicing. And third, redirecting splicing using duplex RNAs provides an alternative to using antisense oligonucleotides that may prove advantageous. Modulation of alternative splicing by small RNAs offers another layer to the subtle pattern of RNA-mediated regulation that exists inside cells.

APOB.

Apolipoprotein B (APOB) is a major structural apolipoprotein in the LDL, VLDL, IDL, Lp(a) and chylomicron lipoprotein particles. APOB has two natural isoforms: the full-length APOB100 isoform and the C-terminally truncated APOB48. Full length APOB100 is synthesized and secreted in liver and then assembled into LDL, VLDL, IDL and Lp(a). Enhancing levels of APOB100-containing particles LDL and Lp(a) are related with atherogenesis. Down-regulation of APOB100 is expected to lower circulating LDL and cholesterol levels. Therefore, APOB100 is a major therapeutic target for atherogenesis treatment.

ApoB pre-mRNA consists of 29 constitutively-spliced exons. Redirection of alternative splicing of APOB by antisense oligonucleotides (ASO) could produce truncated APOB and lower the level of full length APOB100. Based on the reports that patients with hypobetalipoproteinemia have lower cholesterol and LDL levels due to the mutation on APOB which results in a C-terminally truncated isoform of APOB, exon27 of APOB is chosen as skipping target when treated by ASO to produce a similar C-terminally truncated isoform APOBΔE27.

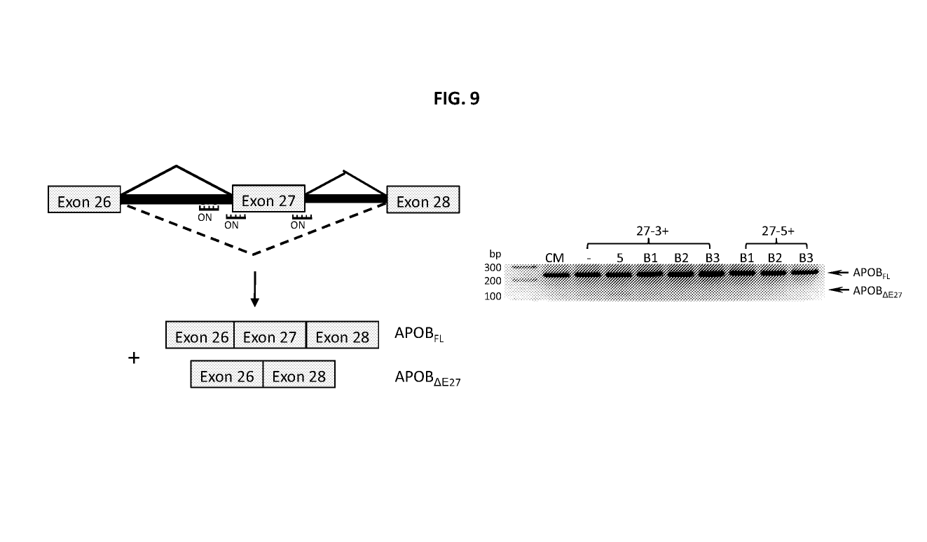

FIG. 9 shows mouse hepatocyte BNL CL.2 (ATCC, TIB-73™) cells treated with RNA duplex alone or duplex mixture (final concentration 100 nM; Table 4). RT-PCR detects the truncated isoform APOBΔE27. After cotransfection of RNA duplexes 27-3 together with 27-5 or with B1, the splicing of full length APOB was redirected and produced exon 27 skipped spliceform APOBΔE27, which will lower circulating LDL and cholesterol levels. However, RNA duplexes control CM, 27-3 alone could not alternate the splicing. Neither did cotransfection of 27-3 with B2 or B3, 27-5 with B1, B2 or B3.

TABLE 1
Designed siRNAs targeting the intron region
of luciferase pre-mRNA

| | | Position | |
|---|---|---|---|
| | | of | Tm |
| siRNA | Sequence and SEQ ID NO: | mismatch | (° C.) |
| 701 | CUUACCUCAGUUACAAUUU (11) | | |
| 703 | CUCUUACCUCAGUUACAAU (12) | | |
| 705 | ACCUCUUACCUCAGUUACA (13) | | 60.2 |
| 707 | AAACCUCUUACCUCAGUUA (14) | | |
| 709 | UGAAACCUCUUACCUCAGU (15) | | |
| 701M11 | CUUACCUCAGAUACAAUUU (16) | 11 | 45.7 |
| 703M11 | CUCUUACCUCUGUUACAAU (17) | 11 | 49.9 |
| 705M11 | ACCUCUUACCACAGUUACA (18) | 11 | 72 |
| 707M11 | AAACCUCUUAGCUCAGUUA (19) | 11 | 55.1 |
| 709M11 | UGAAACCUCUAACCUCAGU (20) | 11 | 58.3 |
| 709M6 | UGAAAGCUCUUACCUCAGU (21) | 6 | |
| 709MMM | UGAAACCUGAAACCUCAGU (22) | 9, | |
| | | 10, 11 | |
| CM | GCUAUACCAGCGUCGUCAU (23) | | 80.0 |

All sequences are listed from 5′ to 3′ and have dTdT at the 3′ terminus. Only the guide strand of siRNA is shown. Mismatched bases are underlined and in bold letters. CM is a non-complementary negative control siRNA.

TABLE 2
Designed siRNAs targeting SMN2 pre-mRNA

| | | Position of | |
|---|---|---|---|
| | | mismatch 9 | Targeted |
| siRNA | Sequence and SEQ ID NO. | (if any) | region |
| | Fully complementary Duplexes | | |
| I82-I64FC | UAGCUUUAUAUGGAUGUUA (24) | — | ISS, intron 6 |
| I13-E06FC | UAAAACCCUGUAAGGAAAA (25) | — | Intron 6/exon 7 |
| I10-E09FC | UCUAAAACCCUGUAAGGAA (26) | — | Intron 6/exon 7 |
| I3-E16FC | GAUUUUGUCUAAAACCCUG (2) | — | ESS, exon 7 |
| E01-E19FC | UUUGAUUUUGUCUAAAACC (1) | — | ESS, exon 7 |
| I6-I24FC | CUUUCAUAAUGCUGGCAGA (27) | — | ISS, intron 7 |
| I9-I27FC | UCACUUUCAUAAUGCUGGC (28) | — | ISS, intron 7 |
| I86-I104FC | CUUUCUAACAUCUGAACUU (29) | — | ISS, intron 7 |
| I89-I107FC | CAACUUUCUAACAUCUGAA (30) | — | ISS, intron 7 |
| | Mismatch-containing duplexes | | |
| I82-I64 | UAGCUUUAUUUGGAUGUUA (31) | 10 | ISS, intron 6 |
| I112-I94 | GUUUCACAACACAUUUUAC (32) | 10 | ISS, intron 6 |
| I5-E14 | UUUUGUCUAAUACCCUGUA (33) | 11 | ESS, exon 7 |
| I3-E16 | GAUUUUGUCAAAAACCCUG (4) | 10 | ESS, exon 7 |
| E01-E19 | UUUGAUUUUGACUAAAACC (3) | 11 | ESS, exon 7 |
| E25-E42 | AGGAAUGUGACCACCUUCC (34) | 11 | Exon 7 |
| I6-I24 | CUUUCAUAAAGCUGGCAGA (35) | 10 | ISS, intron 7 |
| I9-I27 | UCACUUUCAAAAUGCUGGC (36) | 10 | ISS, intron 7 |
| I86-I104 | CUUUCUAACUUCUGAACUU (37) | 10 | ISS, intron 7 |
| | Noncomplementary duplex | | |
| CM | GCUAUACCAGCGUCGUCAU (23) | | |

All sequences are listed from 5′ to 3′ and have dTdT at the 3′ terminus. Only the guide strand of siRNA is shown. Mismatched bases are underlined and in bold letters. CM is a non-complementary negative control siRNA. ISS, intronic splicing silencer; ESS, exonic splicing silencer.

TABLE 3
Designed siRNAs targeting DMD pre-mRNA

| | | Position | |
|---|---|---|---|
| | | of | |
| | Sequence and | mismatch | Targeted |
| siRNA | SEQ ID NO: | (if any) | region |
| E51-64 | GGAAGAUGGCUUUUCUAGU (38) | 11 | Exon 51 |
| E51-66 | AAGGAAGAUGCCAUUUCUA (39) | 11 | Exon 51 |
| E51-68 | UCAAGGAAGAAGGCAUUUC (40) | 11 | Exon 51 |
| E51- | UCAAGGAAGUUGGCAUUUC (5) | 10 | Exon 51 |
| 68M10 | | | |
| E51-70 | CAUCAAGGAACAUGGCAUU (6) | 11 | Exon 51 |
| E51-72 | AACAUCAAGGUAGAUGGCA (41) | 11 | Exon 51 |

All sequences are listed from 5′ to 3′ and have dTdT at the 3′ terminus. Only the guide strand of siRNA is shown. Mismatched bases are underlined and in bold letters. CM is a non-complementary negative control siRNA.

TABLE 4
Sequence of RNA duplexes targeting mouse APOB

| | Antisense strand | Sense strand |
|---|---|---|
| | (5′-3′) | (5′-3′) |
| MAopB 27-3 | UACAACUGGAUAGGAGAGAA | UUCUCUCCUAUCCAGUUGUA |
| MAopB 27-5 | GUAAAACUUCCAUACCAAAG | CUUUGGUAUGGAAGUUUUAG |
| MAopB 27-B1 | AUGUCAUUGGUACAUCAUUU | AAAUGAUGUACCAAUGACAU |
| MAopB 27-B2 | AUCAUUUUCUAUGUGUAUGU | ACAUACACAUAGAAAAUGAU |
| MAopB 27-B3 | GGUACAUCAUUUUCUAUGUG | CACAUAGAAAAUGAUGUACG |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference.
U.S. Pat. No. 4,529,561
U.S. Pat. No. 4,737,323
U.S. Pat. No. 4,981,957
U.S. Pat. No. 5,118,800
U.S. Pat. No. 5,319,080
U.S. Pat. No. 5,359,044
U.S. Pat. No. 5,393,878
U.S. Pat. No. 5,446,137
U.S. Pat. No. 5,466,786
U.S. Pat. No. 5,514,785
U.S. Pat. No. 5,519,134
U.S. Pat. No. 5,567,811
U.S. Pat. No. 5,576,427
U.S. Pat. No. 5,591,722
U.S. Pat. No. 5,597,909
U.S. Pat. No. 5,610,300
U.S. Pat. No. 5,627,053
U.S. Pat. No. 5,639,873
U.S. Pat. No. 5,646,265
U.S. Pat. No. 5,658,873
U.S. Pat. No. 5,670,633
U.S. Pat. No. 5,700,920
U.S. Pat. No. 5,792,747
U.S. Pat. No. 6,268,490
U.S. Pat. No. 6,525,191
U.S. Pat. No. 6,531,584
U.S. Pat. No. 6,600,032
U.S. Pat. No. 6,670,461
U.S. Pat. No. 6,747,014
U.S. Pat. No. 6,753,423
U.S. Pat. No. 6,770,748
U.S. Pat. No. 6,794,499
U.S. Pat. No. 7,034,133
U.S. Pat. No. 7,053,207
U.S. Patent Publn. 2003/0082807
U.S. Patent Publn. 2003/0207841
U.S. Patent Publn. 2004/0014959
U.S. Patent Publn. 2004/0143114
U.S. Patent Publn. 2004/0171570
U.S. Patent Publn. 2004/0219565
U.S. Patent Publn. 2008/0015162
Aartsma-Rus and van Ommen, RNA, 13:1609-1624, 2007.
Allerson et al., J. Med. Chem., 48:901-904, 2005.
Allo et al., Nat. Struc. Mol. Biol., 16:717-724, 2009.
Alter et al., Nat. Med., 12:175-177, 2006.
Barbaux et al., Nat. Genet., 17:467-470, 1997.
Bauman et al., Oligonucleotides, 19:1-13, 2009.
Binder et al., J. Clin. Endocrinol. Metab., 81:4047-4050, 1996.
Bloomfield, Ann. Rev. Biophys. Bioeng., 10:421-450, 1981.
Braasch et al., Chem. Biol., 8:1-7, 2001.
Buee et al., Brain Res. Brain Res. Rev., 33:95-130, 2000.
Burghes and McGovern, Genes Dev., 24:1574-1579, 2010.
Call et al., Cell, 60:509-520, 1990.
Choung et al., Biochem. Biophys. Res. Commun., 342:919-927, 2006.
Chu et al., Nucleic Acids Res., 38:7736-7748, 2010.
Cogan et al., Hum. Mol. Genet., 6:909-912, 1997.
Cogan et al., J. Clin. Endocrinol. Metab., 79:1261-1265, 1974.
Delaney et al., Nat. Genet., 4:426-431, 1993.
Dominski and Kole, Proc. Natl. Acad. Sci. USA, 90:8673-8677, 1993.
Du et al., Nucleic Acids Res., 33:1671-1677, 2005.
Elayadi et al., Curr. Opinion Invens. Drugs, 2:558-561, 2001.
England et al., Nature, 343(6254):180-182, 1990.
Frieden et al., Nucleic Acids Res., 21:6365-6372, 2003.
Gessler et al., Nature, 343:774-778, 1990.
Gregoriadis, In: Liposome Technology, Vols. 1-3, CRC Press, Boca Raton, Fla., 1993.
Guterstam et al., Biochem. J., 412:307-313, 2008.
Haber et al., Proc. Natl. Acad. Sci. USA, 88:9618-9622, 1991.
Haeberli et al., Nucleic Acids Res., 33:3965-3975, 2005.
Hall et al., Nucleic Acids Res., 32:5991-6000, 2004.
Hall et al., Nucleic Acids Res., 34:2773-2781, 2006.
Hope et al., Biochim. Biophys. Acta, 812:55-65, 1985.
Hoshika et al., Nucleic Acids Res., 32:3815-3825, 2004.
Hua et al., Genes. Dev., 24:1634-1644, 2010.
Janoff, In: Liposomes: Rational Design, Marcel Dekker, NY, 1999.
Jensen et al., Biochem. Soc. Trans., 37:1311-1315, 2009.
Kang et al., Biochemistry, 37:6235-6239, 1998.
Kaur et al., Biochemistry, 45:7347-55, 2006.
Keren et al., Nat. Rev. Genet., 11:345-355, 2010.
Kinali et al., Lancet, 8:918-928, 2009.
Kohsaka et al., Hum. Mutat., 14:466-470, 1999.
Koshkin et al., Tetrahedron, 54:3607-3630, 1998.
Kumar et al., Bioorg. Med. Chem. Lett., 8:2219-2222, 1998.
Lecomte et al., Nucleic Acids Res., 15:6331-6348, 1987.
Li et al., Nat. Med., 11:944-951, 2005.
Liao et al., PLos One, 5:e10563, 2010.
Liu et al., Nat. Cell Biol., 7:719-723, 2005.
Liu et al., Science, 305:1437-1441, 2004.
Lorson et al., Hum. Mol. Genet., 19:R111-R118, 2010.
Lu et al., Mol. Ther., 19:9-15, 2010.
Mann et al., Proc. Natl. Acad. Sci. USA, 98:42-47, 2001.
Melo et al., J. Clin. Endocrinol. Metab., 87:2500-2505, 2002.
Miles et al., Proc. Natl. Acad. Sci. USA, 95:13068-13072, 1998.
Morita et al., Bioorganic Medicinal Chem., 11:2211-2226, 2003.
Moseley et al., J. Clin. Endocrinol. Metab., 87:847-852, 2002.
Noone and Knowles, Respir. Res., 2:328-332, 2001.
Orum et al., Curr. Opinion Mol. Ther., 3:239-243, 2001.
PCT Appln. WO 2005/021570
PCT Appln. WO 2005/115481
PCT Appln. WO 2005/121371
PCT Appln. WO 94/14226
PCT Appln. WO 98/39352
PCT Appln. WO 99/14226
Prakash et al., J. Med. Chem., 48:4247-4253, 2005.
Rand et al., Cell, 123:621-629, 2005.
Remington's Pharmaceutical Sciences, 15^{th} ed., pages 1035-1038 and 1570-1580, Mack Publishing Company, Easton, Pa., 1980.
Remington's Pharmaceutical Sciences, 15^{th} ed., 33:624-652, 1980.
Robb et al., Nat. Struc. Mol. Biol., 12:133-137, 2005.
Sazani et al., Nat. Biotech., 20:1228-1233, 2002.
Sen and Blau, Nat. Cell Biol., 7:633-636, 2005.
Shah, In: Micelles, Microemulsions, and Monolayers: Science and Technology, Marcel Dekker, NY, 1998.
Singh et al., Chem. Commun., 4:455-456, 1998.
Singh et al., J. Org. Chem., 63:10035-10039, 1998.
Soutschek et al., Nature, 432(7014):173-178, 2004.
Strong et al., Hum. Mol. Genet., 2:225-230, 1993.
Szoka and Papahadjopoulos, Proc. Natl. Acad. Sci. USA, 75:4194 4198, 1978.
van Deutekom et al., N. Engl. J. Med., 357:2677-2686, 2007.
Wahlestedt et al., Proc. Natl. Acad. Sci. USA, 97:5633-5638, 2000.
Wang et al., Nature, 456:921-926, 2008.
Watts and Corey, Bioorg. Med. Chem. Lett., 20:3203-3207, 2010.
Wood et al., Brain, 133:957-972, 2010.
Yakota et al., Ann. Neurol., 65:667-676, 2009.
You et al., Nucl. Acids Res., 34:e60, 2006.
Yue et al., Nat. Chem. Biol., 6:621-629, 2010.
Zimmerman et al., Nature, 441:111-114, 2006.

1. A method for modulating splicing of a pre-mRNA comprising contacting a cell that produces a pre-mRNA with alternative splicing with a double-stranded RNA of 15-30 bases that targets a splice junction or exonic or intronic sequences adjacent thereto, wherein (a) said alternative splicing produces an “exon-included” product and said double-stranded RNA contains one or more centrally located mismatches; or (b) said alternative splicing produces and “exon-excluded” product and said double-stranded RNA is fully complementary to a target site.

2. The method of claim 1, said pre-mRNA encodes a protein that is aberrantly spliced in a disease state.

3. The method of claim 2, wherein said protein is the Duchenne muscular dystrophy protein, survival motor neuron 2 protein, the APOB protein, the Bcl-x protein or the insulin receptor protein.

4. The method of claim 2, wherein said double-stranded RNA is 19 to about 21 bases in length.

5. The method of claim 1, wherein said double-stranded RNA comprises one or more chemically-modified bases.

6. The method of claim 5, wherein said chemically-modified base is a 2′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, or a non-natural base comprising nucleotide.

7. The method of claim 5, wherein the guide strand or the passenger strand contains the chemically-modified base.

8. The method of claim 5, wherein the guide and passenger strands both contain at least one chemically-modified base.

9. The method of claim 1, wherein said double-stranded RNA comprises at least 15 bases and said first base mismatch is flanked by at least 7 bases on both the 5′ and 3′ ends of said double-stranded RNA.

10. The method of claim 1, wherein said first base mismatch is in the guide strand.

11. The method of claim 1, wherein said double-stranded RNA further comprises a second base mismatch.

12. The method of claim 11, wherein said second base mismatch is in the passenger strand.

13. The method of claim 11, wherein said first and second base mismatches are in the guide strand.

14-22. (canceled)

23. The method of claim 1, wherein said double-stranded RNA targets a splice junction sequence, or an exonic sequence flanking a splice junction sequence, or an intronic sequence flanking a splice junction sequence.

24-25. (canceled)

26. The method of claim 1, wherein splicing of said pre-mRNA is shifted such that an alternative exon is included.

27. The method of claim 1, wherein splicing of said pre-mRNA is shifted such than an alternative exon is excluded.

28. The method of claim 1, wherein said double-stranded RNA comprises a non-natural internucleotide linkage.

29. The method of claim 28, wherein the non-natural internucleotide linkage is a phosphorothioate linkage, and/or is in a guide strand, passenger strand or both.

30. (canceled)

31. A method for modulating splicing, in a subject, of an pre-mRNA encoding a disease protein and having alternative splicing comprising administering to said subject a double-stranded RNA of 15-30 bases that targets a splice junction or exonic or intronic sequences adjacent thereto, wherein (a) said alternative splicing produces an “exon-included” product and said double-stranded RNA contains one or more centrally located mismatches; or (b) said alternative splicing produces and “exon-excluded” product and said double-stranded RNA is fully complementary to a target site.

32-65. (canceled)

66. A composition of matter comprising a double-stranded RNA of 15-30 bases that that targets a splice junction or exonic or intronic sequences adjacent thereto in a pre-mRNA, wherein (a) said alternative splicing produces an “exon-included” product and said double-stranded RNA contains one or more centrally located mismatches; or (b) said alternative splicing produces and “exon-excluded” product and said double-stranded RNA is fully complementary to a target site.

67-91. (canceled)
